(19) United States
(12) Patent Application Publication
(10) Pub. No.: US 2025/0170037 A1
(43) Pub. Date: May 29, 2025

(54) EYE MAKEUP COMPOSITION BASED ON A VOLATILE MIXTURE OF ALKANES

(71) Applicant: BIOSYNTHIS, Saint Cyr Sous Dourdan (FR)

(72) Inventor: Thierry BERNOUD, Saint Cyr Sous Dourdan (FR)

(73) Assignee: BIOSYNTHIS, Saint Cyr Sous Dourdan (FR)

(21) Appl. No.: 18/843,501

(22) PCT Filed: Mar. 3, 2023
(86) PCT No.: PCT/EP2023/055482

§ 371 Date: Dec. 2, 2024

(30) Foreign Application Priority Data
Mar. 3, 2022 (FR) 2201874

(51) Int. Cl. A61K 8/31; A61Q 1/10

(52) U.S. Cl. CPC A61K 8/31; A61Q 1/10

(57) ABSTRACT

An eye makeup composition characterized in that it includes a volatile mixture of alkanes, the volatile mixture of alkanes including: a) from 20 to 100% of a fraction A which includes at least one C≤10 branched alkane; b) from 0 to 80% of a fraction B which includes at least one C>10 linear or branched alkane; and characterized in that the volatile mixture of alkanes has a flash point of less than 55° C. measured in accordance with standard ASTM D93 and represents a weight percentage greater than or equal to 10% relative to the total weight of the composition. Also, the process for preparing the composition.

This application concerns the technical field of cosmetics.

More specifically, the invention relates to an eye makeup composition comprising a volatile mixture of alkanes.

Even more specifically, the invention relates to a mascara composition or an eyeliner composition comprising a volatile mixture of alkanes.

As part of this application, a number of definitions must be given.

An “alkane” is a saturated hydrocarbon consisting only of carbon and hydrogen atoms linked together by simple covalent bonds whose general formula is CₙH₂ₙ₊₂.

A “linear alkane” is an alkane in which each carbon atom is bonded to a maximum of two carbon atoms.

A “branched alkane” is an alkane in which some carbon atoms are linked to three or even four carbon atoms.

A “Cₓ alkane” is an alkane made up of x carbon atoms, which alkane can be linear or branched. For example, dodecane is an alkane consisting of 12 carbon atoms or C₁₂.

Commonly, a “volatile mixture of alkanes” is a mixture of alkanes having a flash point less than or equal to 95° C. measured according to the ATSM D93 standard.

For the purposes of the present invention, the term “volatile mixture of alkanes” means a mixture of alkanes having a flash point less than or equal to 55° C. measured according to the ATSM D93 standard.

The flash point measured according to ASTM D93 is called the “flash point”. The unit of flash point measured according to ASTM D93 is degrees Celsius (° C.). It can also be an average of values, each value being obtained by means of a measurement according to the ASTM D93 standard.

The term “mass percentage” refers to the ratio of the mass of a first compound to the total mass of a mixture of compounds or composition, reduced to a percentage. For example, if 10 grams of a compound y are present in a mixture z having a total mass of 100 grams, then the mass percentage of y in z is 10%.

A “bio-based” compound or organic composition is defined as one in which the organic carbon present in the compound or composition is 100% of plant origin (¹⁴C) by radiocarbon analysis according to one of the following standards: ASTM D6866, EN 16640 or EN 16785-1.

The “index of naturalness” of an organic composition is the percentage of organic carbon of plant origin (¹⁴C) determined by radiocarbon analysis according to one of the following standards: ASTM D6866, EN 16640 or EN 16785-1.

For the purposes of the present invention, the OECD 301F method makes it possible to assess the biodegradability of a substance by a manometric respirometry test.

Since the appearance of isododecane in cosmetics in the 1990s, its use has expanded considerably in all areas of cosmetics, from skin care products to makeup products such as mascara compositions, long-lasting lipstick, and hair products such as serums or anti-frizz dry oils.

In some of these products, isododecane can represent up to 60% by mass percentage of the composition of these cosmetic formulas.

Long-lasting makeup now represents around 25% of the global makeup market: isododecane is a key component because it allows for long-lasting, transfer-free and water-resistant performance, while maintaining optimal comfort. This market was developed from this essential ingredient, isododecane, from petrochemicals.

However, with oil resources being limited and the oil market being fluctuating and polluting, meeting the demand for isododecane or being able to provide at least equivalent properties in cosmetic formulations represents a strategic challenge for the cosmetic industry.

To address such a challenge, a first approach is to develop a process for producing isododecane that does not use petroleum raw materials.

Recently, GLOBAL BIOENERGIES disclosed in its application WO2020188033, the production of isobutene by a biological process using a genetically modified organism such as the modified E. Coli bacteria.

The isobutene gas obtained is then converted into isododecane by a solvent phase oligomerization reaction followed by a hydrogenation step according to patent application WO2021228824.

This reaction, known in the chemical industry, has the disadvantage of forming by-products and offering a low yield, which raises questions about the viability of this approach in the long term in terms of volume and cost.

Nevertheless, more than €100 million was raised by GLOBAL BIOENERGIES for the development of such a process between 2008 and early 2021, the date of release of the first batch of cosmetic-grade isododecane.

This long-term economic investment for the production of isododecane from natural raw materials is remarkable.

Indeed, a second strategy, consisting of reducing the quantity of isododecane present in current cosmetic products or even substituting it, is a challenge of a completely different magnitude for the cosmetics industry.

Furthermore, finding a biodegradable substitute for isododecane would be a major advance from an environmental point of view, as mentioned in patent application EP3349720, such a compound being able to represent up to a mass percentage of 60% of the cosmetic composition.

Research was thus carried out in order to identify compounds likely to offer a satisfactory alternative to isododecane, these compounds having to have similar volatility characteristics and similar behavior in a formulation, in particular in terms of sensory properties such as the ability to spread on the skin, the sensation to the touch, or even the “non-greasy” character of the film obtained.

Thus, there remains a need, from an economic and ecological point of view, to have a cosmetic excipient capable of substituting isododecane, in particular as an excipient to be included in cosmetic formulations.

Surprisingly, it was demonstrated by the Applicant that such an excipient could be obtained.

This excipient is a volatile mixture of alkanes as previously defined.

Preferably, this volatile mixture of alkanes is obtained from fatty alcohols by a Guerbet alcohol reaction followed by a dehydration and hydrogenation reaction. This synthesis route advantageously makes it possible to obtain a substitute excipient for isododecane, from raw materials of natural origin using an economically viable industrial process in relation to market needs.

An excipient of “natural origin” means an excipient that is obtained directly from the earth, soil, water, atmosphere or from plants, via, where appropriate, one or more physical processes, such as, for example, grinding, refining, distillation, purification or filtration.

An excipient of “natural origin” is understood to mean an excipient which has undergone at least one chemical or industrial treatment, resulting in modifications which do not affect the essential qualities of this excipient and/or an excipient comprising mainly natural constituents which have or have not undergone transformations, as indicated above.

As a non-limiting example of additional chemical or industrial treatments resulting in modifications that do not affect the essential qualities of a natural compound, we can mention those authorized by control organizations such as Ecocert or Cosmos.

For the purposes of the invention, the term “synthetic or artificial excipient” means an ingredient which does not meet the definitions of natural or naturally-derived excipients indicated above.

Moreover, such an excipient is biodegradable according to the OECD 301F method unlike isododecane (CAS 13475-82-6).

Advantageously, the excipient according to the present invention has a biodegradability of at least 50% according to the OECD 301F method.

It has also been demonstrated according to the invention that the incorporation of this cosmetic excipient into cosmetic formulations, in particular those for the eyes such as mascara or eyeliner, had the effect of providing the said cosmetic formulation with remarkable cosmetic qualities in addition to excellent stability of these formulations, such as ease of application, water resistance, good, and a more intense color.

Furthermore, the incorporation of this cosmetic excipient has made it possible to obtain cosmetic eye makeup formulations that can present an index of naturalness greater than 90%.

The invention relates to an eye makeup composition, characterized in that it comprises a volatile mixture of alkanes, the said volatile mixture of alkanes comprising:
a) From 20 to 100% by mass of a fraction A which comprises at least one branched C≤10 alkane;
b) From 0 to 80% by mass of a fraction B which comprises at least one linear or branched C>10 alkane;
   and in that the said volatile mixture of alkanes has a flash point below 55° C. measured according to ASTM D93 and represents a mass percentage greater than or equal to 10% relative to the total mass of the composition.

The invention also relates to an eye makeup composition, characterized in that it comprises a volatile mixture of alkanes, the said volatile mixture of alkanes comprising:
a) From 20 to 100% by mass of a fraction A which comprises at least one branched C≤10 alkane;
b) From 0 to 80% by mass of a fraction B which comprises at least one linear or branched C>10 alkane;
   and in that the said volatile mixture of alkanes has a flash point below 55° C. measured according to ASTM D93 and represents a mass percentage greater than or equal to 25% relative to the total mass of the composition.

The composition of the eye makeup according to the invention is characterized in that it comprises a mass percentage of less than or equal to 15% of isododecane relative to the total mass of the composition.

The composition of the eye makeup according to the invention is characterized in that it comprises a mass percentage of less than or equal to 10% of isododecane relative to the total mass of the composition.

Preferably, the composition of the eye makeup according to the invention is characterized in that it comprises a mass percentage of less than or equal to 5% of isododecane relative to the total mass of the composition.

Preferably, the composition of the eye makeup according to the invention is characterized in that it is free of isododecane.

In one embodiment, the composition of the eye makeup according to the invention is characterized in that it is free of linear C₁₀ alkanes.

In one embodiment, the composition of the eye makeup according to the invention is characterized in that it is free of volatile silicones.

The composition of the eye makeup according to the invention is characterized in that it is either a mascara composition or an eyeliner composition.

Preferably, the composition of the eye makeup according to the invention is a mascara composition.

Traditionally, according to the book entitled “Conception des produits cosmétiques: la formulation”, by Anne-Marie PENSE-LHERITIER published in 2014 by LAVOISIER, there are mainly two types of mascara formulations:
Continuous water-phase mascaras are also called “water-based mascara”
Continuous oil phase mascaras are also called “waterproof mascara”

In the remainder of this invention, we will focus mainly on this second category of mascara formulation.

Continuous oil phase mascaras rely mainly on the use of wax or continuous oil phase emulsion. The aim is to form a continuous film on the surface of the eyelash, a film that is perfectly water-resistant.

To do this, the mascara composition includes in particular an oily phase and a pigmentary part and this composition may optionally include an aqueous phase.

The mascara composition according to the present invention is characterized in that it comprises a mass percentage of less than or equal to 10% of water relative to the total mass of the composition.

Preferably, the mascara composition according to the present invention is free of an aqueous phase.

The fatty phase includes in particular, volatile solvents, oils, surfactants, waxes, gelling agents, film-forming polymers, and preservatives.

The pigment part includes water-soluble dyes, fat-soluble dyes, pigments, pearlescent agents and glitters.

Obtaining a mascara is done by finding a subtle balance between these different ingredients.

These ingredients can be supplemented with various additives providing specific properties: polymers, absorbent powders, stabilizing additives such as antioxidants, perfume and active ingredients where appropriate.

Regarding eyeliners, their composition is similar to that of the mascaras developed above.

The various ingredients of the composition of the eye makeup according to the invention are detailed below:

Typically, volatile solvents used in the cosmetic industry are volatile silicones or volatile isoparaffins such as isododecane.

For the purposes of the present invention, the volatile solvent is defined as a “volatile mixture of alkanes”.

In one embodiment, the composition of the eye makeup according to the invention is characterized in that the flash point of the said volatile mixture of alkanes is less than or equal to 50° C.

Preferably, the composition of the eye makeup according to the invention is characterized in that the flash point of the said volatile mixture of alkanes is between 45 and 50° C.

Preferably, the composition of the eye makeup according to the invention is characterized in that the flash point of the said volatile mixture of alkanes is less than or equal to 45° C.

Even more preferably, the composition of the eye makeup according to the invention is characterized in that the flash point of the said volatile mixture of alkanes is between 40 and 45° C.

Advantageously, the composition of the eye makeup according to the invention is characterized in that the flash point of the said volatile mixture of alkanes is less than or equal to 40° C.

In one embodiment, the composition of the eye makeup according to the invention is characterized in that the flash point of the said volatile mixture of alkanes is between 35 and 40° C.

In one embodiment, the “volatile mixture of alkanes” is a mixture of alkanes free of alkanes having a carbon number greater than 16.

Preferably, the “volatile mixture of alkanes” is a mixture of alkanes free of alkanes having a carbon number greater than 15.

Preferably, the “volatile mixture of alkanes” is a mixture of alkanes free of alkanes whose number of carbon atoms is greater than 14.

In one embodiment, the composition of the eye makeup according to the invention is characterized in that the said volatile mixture of alkanes has an index of naturalness greater than or equal to 60% carbon 14 (¹⁴C) determined according to one of the following standards ASTM D6866, EN 16640 or EN 16785-1.

In one embodiment, the composition of the eye makeup according to the invention is characterized in that the said volatile mixture of alkanes has an index of naturalness greater than or equal to 70% carbon 14 (¹⁴C) determined according to one of the following standards ASTM D6866, EN 16640 or EN 16785-1.

Preferably, the composition of the eye makeup according to the invention is characterized in that the said volatile mixture of alkanes has an index of naturalness greater than or equal to 75% carbon 14 (¹⁴C) determined according to one of the following standards ASTM D6866, EN 16640 or EN 16785-1.

Preferably, the composition of the eye makeup according to the invention is characterized in that the said volatile mixture of alkanes has an index of naturalness greater than or equal to 85% carbon 14 (¹⁴C) determined according to one of the following standards ASTM D6866, EN 16640 or EN 16785-1.

Preferably, the composition of the eye makeup according to the invention is characterized in that the said volatile mixture of alkanes has an index of naturalness greater than or equal to 90% carbon 14 (¹⁴C) determined according to one of the following standards ASTM D6866, EN 16640 or EN 16785-1.

Advantageously, the volatile mixture of alkanes present in the composition according to the invention has an index of naturalness of 100% carbon 14 (¹⁴C) determined according to one of the following standards ASTM D6866, EN 16640 or EN 16785-1.

In one embodiment, the composition of the eye makeup according to the invention is characterized in that the said volatile mixture of alkanes is obtained from raw materials of plant, bacterial or animal origin.

In a preferred embodiment, the composition of the eye makeup according to the invention is characterized in that the said volatile mixture of alkanes is obtained from raw materials of plant origin.

In one embodiment, the composition of the eye makeup according to the invention is characterized in that the volatile mixture of alkanes has a biodegradability of at least 50% according to the OECD 301F method.

In one embodiment, the composition of the eye makeup according to the invention is characterized in that the volatile mixture of alkanes has a biodegradability of at least 60% according to the OECD 301F method.

In one embodiment, the composition of the eye makeup according to the invention is characterized in that the volatile mixture of alkanes has a biodegradability of between 50 and 80% according to the OECD 301F method.

In one embodiment, the composition of the eye makeup according to the invention is characterized in that the volatile mixture of alkanes has a biodegradability of between 50 and 70% according to the OECD 301F method.

In one embodiment, the composition of the eye makeup according to the invention is characterized in that the volatile mixture of alkanes has a biodegradability of between 50 and 60% according to the OECD 301F method.

In one embodiment, the composition of the eye makeup according to the invention is characterized in that the volatile mixture of alkanes has a biodegradability of between 60 and 70% according to the OECD 301F method.

In one embodiment, the composition of the eye makeup according to the invention is characterized in that the volatile mixture of alkanes has a biodegradability of between 70 and 80% according to the OECD 301F method.

In one embodiment, the composition of the eye makeup according to the invention is characterized in that the mass percentage of the said volatile mixture of alkanes is at least 10% relative to the total mass of the composition.

In one embodiment, the composition of the eye makeup according to the invention is characterized in that the mass percentage of the said volatile mixture of alkanes is at least 30% relative to the total mass of the composition.

In one embodiment, the composition of the eye makeup according to the invention is characterized in that the mass percentage of the said volatile mixture of alkanes is between 10 and 80% relative to the total mass of the composition.

In one embodiment, the composition of the eye makeup according to the invention is characterized in that the mass percentage of the said volatile mixture of alkanes is between 25 and 80% relative to the total mass of the composition.

In one embodiment, the composition of the eye makeup according to the invention is characterized in that the mass percentage of the said volatile mixture of alkanes is between 30 and 80% relative to the total mass of the composition.

In one embodiment, the composition of the eye makeup according to the invention is characterized in that the mass percentage of the said volatile mixture of alkanes is between 40 and 80% relative to the total mass of the composition.

In one embodiment, the composition of the eye makeup according to the invention is characterized in that the mass percentage of the said volatile mixture of alkanes is between 30 and 60% relative to the total mass of the composition.

In one embodiment, the composition of the eye makeup according to the invention is characterized in that the mass percentage of the said volatile mixture of alkanes is between 25 and 50% relative to the total mass of the composition.

In one embodiment, the composition of the eye makeup according to the invention is characterized in that the mass percentage of the said volatile mixture of alkanes is between 10 and 30% relative to the total mass of the composition.

In one embodiment, the composition of the eye makeup according to the invention is characterized in that the mass percentage of the said volatile mixture of alkanes is between 40 and 60% relative to the total mass of the composition.

In one embodiment, the composition of the eye makeup according to the invention is characterized in that the mass percentage of the said volatile mixture of alkanes is between 60 and 80% relative to the total mass of the composition.

Fraction A of the said volatile mixture of alkanes is selected from the group comprising branched C₈ alkanes, branched C₉ alkanes, branched C₁₀ alkanes, and mixtures thereof.

Fraction A of the said volatile mixture of alkanes comprises at least one branched C≤10 alkane.

In one embodiment, the composition of the eye makeup according to the invention is characterized in that at least one branched C≤10 alkane of the said volatile mixture of alkanes is at least one branched C₈ alkane.

In one embodiment, the composition of the eye makeup according to the invention is characterized in that at least one branched C≤10 alkane of the said volatile mixture of alkanes is chosen from the group comprising branched C₉ alkanes, branched C₁₀ alkanes, and mixtures thereof.

In one embodiment, the composition of the eye makeup according to the invention is characterized in that at least one branched C≤10 alkane of the said volatile mixture of alkanes is a branched C₉ alkane.

In one embodiment, the composition of the eye makeup according to the invention is characterized in that the at least one branched C≤10 alkane of the said volatile mixture of alkanes is at least one branched C₉ alkane selected from the group consisting of trimethylhexanes such as for example 2,2,4-trimethylhexane (CAS 16747-26-5), 2,2,5-trimethylhexane (CAS 3522-94-9), 2,2,3-trimethylhexane (CAS 16747-25-4), 2,4,4-trimethylhexane (CAS 16747-30-1), 2,3,3-trimethylhexane (CAS 16747-28-1), 3,3,4-trimethylhexane (CAS 16747-31-2), 2,3,5-trimethylhexane (CAS 1069-53-0), 2,3,4-trimethylhexane (CAS 921-47-1).

For the purposes of the present invention, the term “trimethylhexanes” means all C₆ alkanes branched by three methyl groups.

In one embodiment, the composition of the eye makeup according to the invention is characterized in that at least one branched C≤10 alkane of the said volatile mixture of alkanes is a trimethylhexane.

In one embodiment, the composition of the eye makeup according to the invention is characterized in that at least one branched C≤10 alkane of the said volatile mixture of alkanes is 2,2,4-trimethylhexane (CAS 16747-26-5).

In one embodiment, the composition of the eye makeup according to the invention is characterized in that at least one branched C≤10 alkane of the said volatile mixture of alkanes is 2,3,5-trimethylhexane (CAS 1069-53-0).

In one embodiment, the composition of the eye makeup according to the invention is characterized in that at least one branched C≤10 alkane of the said volatile mixture of alkanes is 2,3,4 trimethylhexane (CAS 921-47-1).

In one embodiment, the composition of the eye makeup according to the invention is characterized in that at least one branched C≤10 alkane of the said volatile mixture of alkanes is a branched C₁₀ alkane.

In one embodiment, the composition of the eye makeup according to the invention is characterized in that at least one branched C≤10 alkane of the said volatile mixture of alkanes is a branched C₁₀ alkane selected from the group consisting of: 2-methylnonane (CAS 871-83-0), 4-methylnonane (CAS 17301-94-9), 3-methylnonane (CAS 5911-04-6), 3-ethyloctane (CAS 5881-17-4), 2,2-dimethyloctane (CAS 15869-87-1), 2,3-dimethyloctane (CAS 7146-60-3), 2,5-dimethyloctane (CAS 15869-89-3), 3,5-dimethyloctane (CAS 15869-93-9), 4-propylheptane (CAS 3178-29-8), 3-ethyl-2-methylheptane (CAS 14676-29-0), 2,3,6-trimethylheptane (CAS 4032-93-3), 3,3,4-trimethylheptane (CAS 20278-87-9), 2,3,4-trimethylheptane (CAS 52896-95-4), 2,2,4-trimethylheptane (CAS 14720-74-2), 3,3-diethylhexane (CAS 17302-02-2), 2,2,3,3-tetramethylhexane (CAS 13475-81-5), 3-ethyl-2,2,3-trimethylpentane (CAS 52897-17-3), 2,2,3-trimethylheptane (CAS 52896-92-1), 2,3,5-trimethylheptane (CAS 20278-85-7), 3,3-diethylhexane (CAS 17302-02-2), 2,2,3,3-tetramethylhexane (CAS 13475-81-5), 3-ethyl-2,2,3-trimethylpentane (CAS 52897-17-3), 3,3,5-trimethylheptane (CAS 7154-80-5), 2,5,5-trimethylheptane (CAS 1189-99-7), 2,4,6-trimethylheptane (CAS 2613-61-8), 3,4,5-trimethylheptane (CAS 20278-89-1), 2,2,6-trimethylheptane (CAS 1190-83-6), 2,4,5-trimethylheptane (CAS 20278-84-6), 2,4,4-trimethylheptane (CAS 4032-92-2), 2,3,3-trimethylheptane (CAS 52896-93-2), 3,4,4-trimethylheptane (CAS 20278-88-0), and mixtures thereof.

For the purposes of the present invention, the term “trimethylheptanes” means all C₇ alkanes branched by three methyl groups.

In one embodiment, the composition of the eye makeup according to the invention is characterized in that at least one branched C≤10 alkane of the said volatile mixture of alkanes is a branched C₁₀ alkane selected from the group consisting of trimethylheptanes such as, for example, 2,2,3-trimethylheptane (CAS 52896-92-1), 2,3,5-trimethylheptane (CAS 20278-85-7), 2,3,6-trimethylheptane (CAS 4032-93-3), 3,3,4-trimethylheptane (CAS 20278-87-9), 2,3,4-trimethylheptane (CAS 52896-95-4), 2,2,4-trimethylheptane (CAS 14720-74-2, 3,3,5-trimethylheptane (CAS 7154-80-5), 2,5,5-trimethylheptane (CAS 1189-99-7), 2,4,6-trimethylheptane (CAS 2613-61-8), 3,4,5-trimethylheptane (CAS 20278-89-1), 2,2,6-trimethylheptane (CAS 1190-83-6), 2,4,5-trimethylheptane (CAS 20278-84-6), 2,4,4-trimethylheptane (CAS 4032-92-2), 2,3,3-trimethylheptane (CAS 52896-93-2), 3,4,4-trimethylheptane (CAS 20278-88-0), and mixtures thereof.

The invention also relates to an eye makeup composition comprising a volatile mixture of alkanes as previously defined, characterized in that at least one branched C≤10 alkane of the said volatile mixture of alkanes is a trimethylheptane.

The invention also relates to an eye makeup composition comprising a volatile mixture of alkanes as previously defined, characterized in that at least one branched C≤10 alkane of the said volatile mixture of alkanes is 2,3,6-trimethylheptane (CAS 4032-93-3).

In one embodiment, the composition of the eye makeup according to the invention is characterized in that at least one branched C≤10 alkane of the said volatile mixture of alkanes is 2,3,5-trimethylheptane (CAS 20278-85-7).

In one embodiment, the composition of the eye makeup according to the invention is characterized in that at least one branched C≤10 alkane of the said volatile mixture of alkanes is 2,2,3-trimethylheptane (CAS 52896-92-1).

In one embodiment, the composition of the eye makeup according to the invention is characterized in that at least one branched C≤10 alkane of the said volatile mixture of alkanes is 3,3,4-trimethylheptane (CAS 20278-87-9).

In one embodiment, the composition of the eye makeup according to the invention is characterized in that at least one branched C≤10 alkane of the said volatile mixture of alkanes is 2,3,4-trimethylheptane (CAS 52896-95-4).

In one embodiment, the composition of the eye makeup according to the invention is characterized in that at least one branched C≤10 alkane of the said volatile mixture of alkanes is 2,2,4-trimethylheptane (CAS 14720-74-2)

In one embodiment, the composition of the eye makeup according to the invention is characterized in that at least one branched C≤10 alkane of the said volatile mixture of alkanes is 2,2,3-trimethylheptane (CAS 52896-92-1).

In one embodiment, the composition of the eye makeup according to the invention is characterized in that at least one branched C≤10 alkane of the said volatile mixture of alkanes is 3,3,5-trimethylheptane (CAS 7154-80-5).

In one embodiment, the composition of the eye makeup according to the invention is characterized in that at least one branched C≤10 alkane of the said volatile mixture of alkanes is 2,5,5-trimethylheptane (CAS 1189-99-7).

In one embodiment, the composition of the eye makeup according to the invention is characterized in that at least one branched C≤10 alkane of the said volatile mixture of alkanes is 2,4,6-trimethylheptane (CAS 2613-61-8).

In one embodiment, the composition of the eye makeup according to the invention is characterized in that at least one branched C≤10 alkane of the said volatile mixture of alkanes is 3,4,5-trimethylheptane (CAS 20278-89-1).

In one embodiment, the composition of the eye makeup according to the invention is characterized in that at least one branched C≤10 alkane of the said volatile mixture of alkanes is 2,2,6-trimethylheptane (CAS 1190-83-6).

In one embodiment, the composition of the eye makeup according to the invention is characterized in that at least one branched C≤10 alkane of the said volatile mixture of alkanes is 2,4,5-trimethylheptane (CAS 20278-84-6).

In one embodiment, the composition of the eye makeup according to the invention is characterized in that at least one branched C≤10 alkane of the said volatile mixture of alkanes is 2,4,4-trimethylheptane (CAS 4032-92-2).

In one embodiment, the composition of the eye makeup according to the invention is characterized in that at least one branched C≤10 alkane of the said volatile mixture of alkanes is 2,3,3-trimethylheptane (CAS 52896-93-2).

The invention also relates to an eye makeup composition comprising a volatile mixture of alkanes as previously defined, characterized in that at least one branched C≤10 alkane of the said volatile mixture of alkanes is a trimethylheptane and a trimethylhexane.

In one embodiment, the composition of the eye makeup according to the invention is characterized in that the volatile mixture of alkanes is free of branched C₈ alkanes.

In one embodiment, the composition of the eye makeup according to the invention is characterized in that the volatile mixture of alkanes is free of branched C₉ alkanes.

In one embodiment, the composition of the eye makeup according to the invention is characterized in that the mass percentage of fraction A of the volatile mixture of alkanes is at least 20% relative to the total mass of the said volatile mixture of alkanes.

In one embodiment, the composition of the eye makeup according to the invention is characterized in that the mass percentage of fraction A of the volatile mixture of alkanes is greater than 50% relative to the total mass of the said volatile mixture of alkanes.

In one embodiment, the composition of the eye makeup according to the invention is characterized in that the mass percentage of fraction A of the volatile mixture of alkanes is between 20% and 100% relative to the total mass of the said volatile mixture of alkanes.

In one embodiment, the composition of the eye makeup according to the invention is characterized in that the mass percentage of fraction A of the volatile mixture of alkanes is between 20% and 70% relative to the total mass of the said volatile mixture of alkanes.

In one embodiment, the composition of the eye makeup according to the invention is characterized in that the mass percentage of fraction A of the volatile mixture of alkanes is between 51% and 100% relative to the total mass of the said volatile mixture of alkanes.

In one embodiment, the composition of the eye makeup according to the invention is characterized in that the mass percentage of fraction A of the volatile mixture of alkanes is between 30% and 60% relative to the total mass of the said volatile mixture of alkanes.

In one embodiment, the composition of the eye makeup according to the invention is characterized in that the mass percentage of fraction A of the volatile mixture of alkanes is between 40% and 70% relative to the total mass of the said volatile mixture of alkanes.

In one embodiment, the composition of the eye makeup according to the invention is characterized in that the mass percentage of fraction A of the volatile mixture of alkanes is between 70% and 100% relative to the total mass of the said volatile mixture of alkanes.

In one embodiment, the composition of the eye makeup according to the invention is characterized in that the mass percentage of fraction A of the volatile mixture of alkanes is between 30% and 50% relative to the total mass of the said volatile mixture of alkanes.

In one embodiment, the composition of the eye makeup according to the invention is characterized in that the mass percentage of fraction A of the volatile mixture of alkanes is between 40% and 60% relative to the total mass of the said volatile mixture of alkanes.

In one embodiment, the composition of the eye makeup according to the invention is characterized in that the mass percentage of fraction A of the volatile mixture of alkanes is between 50% and 70% relative to the total mass of the said volatile mixture of alkanes.

In one embodiment, the composition of the eye makeup according to the invention is characterized in that the mass percentage of fraction A of the volatile mixture of alkanes is between 70% and 90% relative to the total mass of the said volatile mixture of alkanes.

In one embodiment, the composition of the eye makeup according to the invention is characterized in that the mass percentage of fraction A of the volatile mixture of alkanes is between 80% and 100% relative to the total mass of the said volatile mixture of alkanes.

In one embodiment, the composition of the eye makeup according to the invention is characterized in that the mass percentage of at least one branched C≤10 alkane of the said volatile mixture of alkanes is at least 20% relative to the total mass of the said volatile mixture of alkanes.

In one embodiment, the composition of the eye makeup according to the invention is characterized in that the mass percentage of at least one branched C≤10 alkane of the said volatile mixture of alkanes is greater than 50% relative to the total mass of the said volatile mixture of alkanes.

In one embodiment, the composition of the eye makeup according to the invention is characterized in that the mass percentage of at least one branched C≤10 alkane of the said volatile mixture of alkanes is between 20% and 100% relative to the total mass of the said volatile mixture of alkanes.

In one embodiment, the composition of the eye makeup according to the invention is characterized in that the mass percentage of at least one branched C≤10 alkane of the said volatile mixture of alkanes is between 20% and 70% relative to the total mass of the said volatile mixture of alkanes.

In one embodiment, the composition of the eye makeup according to the invention is characterized in that the mass percentage of at least one branched C≤10 alkane of the said volatile mixture of alkanes is between 51% and 100% relative to the total mass of the said volatile mixture of alkanes.

In one embodiment, the composition of the eye makeup according to the invention is characterized in that the mass percentage of at least one branched C≤10 alkane of the said volatile mixture of alkanes is between 30% and 60% relative to the total mass of the said volatile mixture of alkanes.

In one embodiment, the composition of the eye makeup according to the invention is characterized in that the mass percentage of at least one branched C≤10 alkane of the said volatile mixture of alkanes is between 40% and 70% relative to the total mass of the said volatile mixture of alkanes.

In one embodiment, the composition of the eye makeup according to the invention is characterized in that the mass percentage of at least one branched C≤10 alkane of the said volatile mixture of alkanes is between 70% and 100% relative to the total mass of the said volatile mixture of alkanes.

In one embodiment, the composition of the eye makeup according to the invention is characterized in that the mass percentage of at least one branched C≤10 alkane of the said volatile mixture of alkanes is between 30% and 50% relative to the total mass of the said volatile mixture of alkanes.

In one embodiment, the composition of the eye makeup according to the invention is characterized in that the mass percentage of at least one branched C≤10 alkane of the said volatile mixture of alkanes is between 40% and 60% relative to the total mass of the said volatile mixture of alkanes.

In one embodiment, the composition of the eye makeup according to the invention is characterized in that the mass percentage of at least one branched C≤10 alkane of the said volatile mixture of alkanes is between 50% and 70% relative to the total mass of the said volatile mixture of alkanes.

In one embodiment, the composition of the eye makeup according to the invention is characterized in that the mass percentage of at least one branched C≤10 alkane of the said volatile mixture of alkanes is between 70% and 90% relative to the total mass of the said volatile mixture of alkanes.

In one embodiment, the composition of the eye makeup according to the invention is characterized in that the mass percentage of at least one branched C≤10 alkane of the said volatile mixture of alkanes is between 80% and 100% relative to the total mass of the said volatile mixture of alkanes.

In one embodiment, the composition of the eye makeup according to the invention is characterized in that the mass percentage of 2,3,6-trimethylheptane (CAS 4032-93-3) of at least one branched C≤10 alkane of the said volatile mixture of alkanes is at least 20% relative to the total mass of the said volatile mixture of alkanes.

In one embodiment, the composition of the eye makeup according to the invention is characterized in that the mass percentage of 2,3,6-trimethylheptane (CAS 4032-93-3) of at least one branched C≤10 alkane of the said volatile mixture of alkanes is between 20% and 100% relative to the total mass of the said volatile mixture of alkanes.

In one embodiment, the composition of the eye makeup according to the invention is characterized in that the mass percentage of 2,3,6-trimethylheptane (CAS 4032-93-3) of at least one branched C≤10 alkane of the said volatile mixture of alkanes is between 20% and 70% relative to the total mass of the said volatile mixture of alkanes.

In one embodiment, the composition of the eye makeup according to the invention is characterized in that the mass percentage of 2,3,6-trimethylheptane (CAS 4032-93-3) of at least one branched C≤10 alkane of the said volatile mixture of alkanes is between 50% and 100% relative to the total mass of the said volatile mixture of alkanes.

In one embodiment, the composition of the eye makeup according to the invention is characterized in that the mass percentage of 2,3,6-trimethylheptane (CAS 4032-93-3) of at least one branched C≤10 alkane of the said volatile mixture of alkanes is between 51% and 100% relative to the total mass of the said volatile mixture of alkanes.

In one embodiment, the composition of the eye makeup according to the invention is characterized in that the mass percentage of 2,3,6-trimethylheptane (CAS 4032-93-3) of at least one branched C≤10 alkane of the said volatile mixture of alkanes is between 30% and 60% relative to the total mass of the said volatile mixture of alkanes.

In one embodiment, the composition of the eye makeup according to the invention is characterized in that the mass percentage of 2,3,6-trimethylheptane (CAS 4032-93-3) of at least one branched C≤10 alkane of the said volatile mixture of alkanes is between 40% and 70% relative to the total mass of the said volatile mixture of alkanes.

In one embodiment, the composition of the eye makeup according to the invention is characterized in that the mass percentage of 2,3,6-trimethylheptane (CAS 4032-93-3) of at least one branched C≤10 alkane of the said volatile mixture of alkanes is between 70% and 100% relative to the total mass of the said volatile mixture of alkanes.

In one embodiment, the composition of the eye makeup according to the invention is characterized in that the mass percentage of 2,3,6-trimethylheptane (CAS 4032-93-3) of at least one branched C≤10 alkane of the said volatile mixture of alkanes is between 30% and 50% relative to the total mass of the said volatile mixture of alkanes.

In one embodiment, the composition of the eye makeup according to the invention is characterized in that the mass percentage of 2,3,6-trimethylheptane (CAS 4032-93-3) of at least one branched C≤10 alkane of the said volatile mixture of alkanes is between 40% and 60% relative to the total mass of the said volatile mixture of alkanes.

In one embodiment, the composition of the eye makeup according to the invention is characterized in that the mass percentage of 2,3,6-trimethylheptane (CAS 4032-93-3) of at least one branched C≤10 alkane of the said volatile mixture of alkanes is between 50% and 70% relative to the total mass of the said volatile mixture of alkanes.

In one embodiment, the composition of the eye makeup according to the invention is characterized in that the mass percentage of 2,3,6-trimethylheptane (CAS 4032-93-3) of at least one branched C≤10 alkane of the said volatile mixture of alkanes is between 70% and 90% relative to the total mass of the said volatile mixture of alkanes.

In one embodiment, the composition of the eye makeup according to the invention is characterized in that the mass percentage of 2,3,6-trimethylheptane (CAS 4032-93-3) of at least one branched C≤10 alkane of the said volatile mixture of alkanes is between 80% and 100% relative to the total mass of the said volatile mixture of alkanes.

In one embodiment, the composition of the eye makeup according to the invention is characterized in that the mass percentage of 2,2,4-trimethylhexane (CAS 16747-26-5) of at least one branched C≤10 alkane of the said volatile mixture of alkanes is at least 5% relative to the total mass of the said volatile mixture of alkanes.

In one embodiment, the composition of the eye makeup according to the invention is characterized in that the mass percentage of 2,2,4-trimethylhexane (CAS 16747-26-5) of at least one branched C≤10 alkane of the said volatile mixture of alkanes is at least 10% relative to the total mass of the said volatile mixture of alkanes.

In one embodiment, the composition of the eye makeup according to the invention is characterized in that the mass percentage of 2,2,4-trimethylhexane (CAS 16747-26-5) of at least one branched C≤10 alkane of the said volatile mixture of alkanes is between 5% and 20% relative to the total mass of the said volatile mixture of alkanes.

In one embodiment, the composition of the eye makeup according to the invention is characterized in that the mass percentage of 2,2,4-trimethylhexane (CAS 16747-26-5) of at least one branched C≤10 alkane of the said volatile mixture of alkanes is between 5% and 15% relative to the total mass of the said volatile mixture of alkanes.

In one embodiment, the composition of the eye makeup according to the invention is characterized in that the percentage of 2,2,4-trimethylhexane (CAS 16747-26-5) of at least one branched C≤10 alkane of the said volatile mixture of alkanes is between 10% and 20% relative to the total mass of the said volatile mixture of alkanes.

In one embodiment, the composition of the eye makeup according to the invention is characterized in that the mass percentage of 2,2,4-trimethylhexane (CAS 16747-26-5) of at least one branched C≤10 alkane of the said volatile mixture of alkanes is between 10% and 15% relative to the total mass of the said volatile mixture of alkanes.

In one embodiment, the composition of the eye makeup according to the invention is characterized in that the mass percentage of 2,2,4-trimethylhexane (CAS 16747-26-5) of at least one branched C≤10 alkane of the said volatile mixture of alkanes is between 15% and 20% relative to the total mass of the said volatile mixture of alkanes.

Fraction B of the said volatile mixture of alkanes comprises at least one linear or branched C>10 alkane.

In one embodiment, the composition of the eye makeup according to the invention is characterized in that at least one linear or branched C>10 alkane of said volatile mixture of alkanes is selected from the group comprising C₁₁, C₁₂, C₁₃, C₁₄, C₁₅, C₁₆ alkanes and mixtures thereof.

In one embodiment, the composition of the eye makeup according to the invention is characterized in that at least one linear or branched C>10 alkane of the said volatile mixture of alkanes is selected from the group comprising linear alkanes of C₁₁, C₁₂, C₁₃, C₁₄, C₁₅, and mixtures thereof.

In one embodiment, the composition of the eye makeup according to the invention is characterized in that at least one linear or branched C>10 alkane of the said volatile mixture of alkanes is selected from the group consisting of undecane (CAS 1120-21-4), n-dodecane (CAS 112-40-3), n-tridecane (CAS 629-50-5), n-tetradecane (CAS 629-59-4), n-pentadecane (CAS 629-62-9) and mixtures thereof.

In one embodiment, the composition of the eye makeup according to the invention is characterized in that at least one linear or branched C>10 alkane of the said volatile mixture of alkanes is undecane (CAS 1120-21-4).

In one embodiment, the composition of the eye makeup according to the invention is characterized in that at least one linear or branched C>10 alkane of the said volatile mixture of alkanes is n-dodecane (CAS 112-40-3).

In one embodiment, the composition of the eye makeup according to the invention is characterized in that at least one linear or branched C>10 alkane of the said volatile mixture of alkanes is n-tridecane (CAS 629-50-5).

In one embodiment, the composition of the eye makeup according to the invention is characterized in that at least one linear or branched C>10 alkane of the said volatile mixture of alkanes is the undecane/tridecane mixture marketed under the name CETIOL ULTIMATE by BASF.

In one embodiment, the composition of the eye makeup according to the invention is characterized in that at least one linear or branched C>10 alkane of the said volatile mixture of alkanes is n-tetradecane (CAS 629-59-4).

In one embodiment, the composition of the eye makeup according to the invention is characterized in that at least one linear or branched C>10 alkane of the said volatile mixture of alkanes is selected from the group comprising branched C₁₂ alkanes, branched C₁₃ alkanes, branched C₁₄ alkanes, branched C₁₅ alkanes, branched C₁₆ alkanes, and mixtures thereof.

In one embodiment, the composition of the eye makeup according to the invention is characterized in that at least one linear or branched C>10 alkane of the said volatile mixture of alkanes is a branched C₁₁ alkane.

In one embodiment, the composition of the eye makeup according to the invention is characterized in that at least one linear or branched C>10 alkane of the said volatile mixture of alkanes is a branched C₁₂ alkane.

In one embodiment, the composition of the eye makeup according to the invention is characterized in that at least one linear or branched C>10 alkane of the said volatile mixture of alkanes is a branched C₁₂ alkane selected from the group consisting of:
3-methylundecane (CAS 1002-43-3), 4-methylundecane (CAS 2980-69-0), 5-methylundecane (CAS 1632-70-8), 6-methylundecane (CAS 17302-33-9), 2,4-dimethyldecane (CAS 2801-84-5), 4,4-dimethyldecane (CAS 17312-39-9), 3,5-dimethyldecane (17312-48-0), 2,5-dimethyldecane (CAS 17312-50-4), 2,3-dimethyldecane (17312-44-6), 3,3-dimethyldecane (17302-38-4), 3,7-dimethyldecane (CAS 17312-54-8), 3,4,6-trimethylnonane (CAS 62184-24-1), 3,5,6-trimethylnonane (CAS 62184-26-3), 3,5,7-trimethylnonane (CAS 62184-27-4), 2,5,7-trimethylnonane (CAS 62184-14-9), 2,5,6-trimethylnonane (CAS 62184-13-8), 2,5,7-trimethylnonane (CAS 62184-14-9), 2,5,8-trimethylnonane (CAS 49557-09-7), 3,3,4,5-tetramethyloctane (CAS 62185-21-1), 2,3,4,5-tetramethyloctane (CAS 62199-27-3), 2,2,4,5-tetramethyloctane (CAS 62183-80-6), 2,2,5,7-tetramethyloctane (CAS 62199-19-3), 2,3,4,7-tetramethyloctane (CAS 62199-29-5), 2,4,4,7-tetramethyloctane (CAS 35866-96-7), 3-ethyl-4-methylnonane (CAS 62184-45-6), 3-ethyl-4,5-dimethyloctane (CAS 62183-72-6), 2,5 dimethyl-6-ethyloctane (CAS 62183-50-0), 2,2,4,6,6-pentamethylheptane (CAS 13475-82-6) and their mixtures.

In one embodiment, the composition of the eye makeup according to the invention is characterized in that at least one linear or branched C>10 alkane of the said volatile mixture of alkanes is at least one isododecane.

In one embodiment, the composition of the eye makeup according to the invention is characterized in that at least one linear or branched C>10 alkane of the said volatile mixture of alkanes is 2,2,4,6,6-pentamethylheptane (CAS 13475-82-6).

In one embodiment, the composition of the eye makeup according to the invention is characterized in that at least one linear or branched C>10 alkane of the said volatile mixture of alkanes is a branched C₁₃ alkane.

In one embodiment, the composition of the eye makeup according to the invention is characterized in that at least one linear or branched C>10 alkane of the said volatile mixture of alkanes is a branched C₁₄ alkane.

In one embodiment, the composition of the eye makeup according to the invention is characterized in that at least one linear or branched C>10 alkane of the said volatile mixture of alkanes is a branched C₁₅ alkane.

In one embodiment, the composition of the eye makeup according to the invention is characterized in that at least one linear or branched C>10 alkane of the said volatile mixture of alkanes is at least the hemisqualane marketed under the name NEOSSANCE by AMYRIS.

In one embodiment, the composition of the eye makeup according to the invention is characterized in that at least one linear or branched C>10 alkane of the said volatile mixture of alkanes is at least one C₁₃-C₁₅ mixture marketed under the name EMOGREEN L15 by SEPPIC.

In one embodiment, the composition of the eye makeup according to the invention is characterized in that at least one linear or branched C>10 alkane of the said volatile mixture of alkanes is a branched C₁₆ alkane.

In one embodiment, the composition of the eye makeup according to the invention is characterized in that at least one linear or branched C>10 alkane of the said volatile mixture of alkanes is at least one isohexadecane.

In one embodiment, the composition of the eye makeup according to the invention is characterized in that the mass percentage of fraction B of the volatile mixture of alkanes is between 0 and 80% relative to the total mass of the said volatile mixture of alkanes.

In one embodiment, the composition of the eye makeup according to the invention is characterized in that the mass percentage of fraction B of the volatile mixture of alkanes is between 0 and 50% relative to the total mass of the said volatile mixture of alkanes.

In one embodiment, the composition of the eye makeup according to the invention is characterized in that the mass percentage of fraction B of the volatile mixture of alkanes is between 0 and 30% relative to the total mass of the said volatile mixture of alkanes.

In one embodiment, the composition of the eye makeup according to the invention is characterized in that the mass percentage of fraction B of the volatile mixture of alkanes is at least 30% relative to the total mass of the said volatile mixture of alkanes.

In one embodiment, the composition of the eye makeup according to the invention is characterized in that the mass percentage of fraction B of the volatile mixture of alkanes is between 30% and 80% relative to the total mass of the said volatile mixture of alkanes.

In one embodiment, the composition of the eye makeup according to the invention is characterized in that the mass percentage of fraction B of the volatile mixture of alkanes is between 30% and 60% relative to the total mass of the said volatile mixture of alkanes.

In one embodiment, the composition of the eye makeup according to the invention is characterized in that the mass percentage of fraction B of the volatile mixture of alkanes is between 50% and 80% relative to the total mass of the said volatile mixture of alkanes.

In one embodiment, the composition of the eye makeup according to the invention is characterized in that the mass percentage of fraction B of the volatile mixture of alkanes is between 30% and 50% relative to the total mass of the said volatile mixture of alkanes.

In one embodiment, the composition of the eye makeup according to the invention is characterized in that the mass percentage of fraction B of the volatile mixture of alkanes is between 40% and 60% relative to the total mass of the said volatile mixture of alkanes.

In one embodiment, the composition of the eye makeup according to the invention is characterized in that the mass percentage of fraction B of the volatile mixture of alkanes is between 50% and 70% relative to the total mass of the said volatile mixture of alkanes.

In one embodiment, the composition of the eye makeup according to the invention is characterized in that the mass percentage of fraction B of the volatile mixture of alkanes is between 60% and 80% relative to the total mass of the said volatile mixture of alkanes.

In one embodiment, the composition of the eye makeup according to the invention is characterized in that the mass percentage of fraction B of the volatile mixture of alkanes is between 0% and 10% relative to the total mass of the said volatile mixture of alkanes.

In one embodiment, the composition of the eye makeup according to the invention is characterized in that the mass percentage of fraction B of the volatile mixture of alkanes is between 10% and 20% relative to the total mass of the said volatile mixture of alkanes.

In one embodiment, the composition of the eye makeup according to the invention is characterized in that the mass percentage of fraction B of the volatile mixture of alkanes is between 40% and 50% relative to the total mass of the said volatile mixture of alkanes.

In one embodiment, the composition of the eye makeup according to the invention is characterized in that the mass percentage of fraction B of the volatile mixture of alkanes is between 60% and 70% relative to the total mass of the said volatile mixture of alkanes.

In one embodiment, the composition of the eye makeup according to the invention is characterized in that the mass percentage of fraction B of the volatile mixture of alkanes is between 70% and 80% relative to the total mass of the said volatile mixture of alkanes.

In one embodiment, the composition of the eye makeup according to the invention is characterized in that the mass percentage of at least one linear or branched C>10 alkane of the said volatile mixture of alkanes is between 0 and 80% relative to the total mass of the said volatile mixture of alkanes.

In one embodiment, the composition of the eye makeup according to the invention is characterized in that the mass percentage of at least one linear or branched C>10 alkane of the said volatile mixture of alkanes is between 0 and 50% relative to the total mass of the said volatile mixture of alkanes.

In one embodiment, the composition of the eye makeup according to the invention is characterized in that the mass percentage of at least one linear or branched C>10 alkane of the said volatile mixture of alkanes is between 0 and 30% relative to the total mass of the said volatile mixture of alkanes.

In one embodiment, the composition of the eye makeup according to the invention is characterized in that the mass percentage of at least one linear or branched C>10 alkane of the said volatile mixture of alkanes is at least 30% relative to the total mass of the said volatile mixture of alkanes.

In one embodiment, the composition of the eye makeup according to the invention is characterized in that the mass percentage of at least one linear or branched C>10 alkane of the said volatile mixture of alkanes is at least 50% relative to the total mass of the said volatile mixture of alkanes.

In one embodiment, the composition of the eye makeup according to the invention is characterized in that the mass percentage of at least one linear or branched C>10 alkane of the said volatile mixture of alkanes is between 30% and 80% relative to the total mass of the said volatile mixture of alkanes.

In one embodiment, the composition of the eye makeup according to the invention is characterized in that the mass percentage of at least one linear or branched C>10 alkane of the said volatile mixture of alkanes is between 30% and 60% relative to the total mass of the said volatile mixture of alkanes.

In one embodiment, the composition of the eye makeup according to the invention is characterized in that the mass percentage of at least one linear or branched C>10 alkane of the said volatile mixture of alkanes is between 50% and 80% relative to the total mass of the said volatile mixture of alkanes.

In one embodiment, the composition of the eye makeup according to the invention is characterized in that the mass percentage of at least one linear or branched C>10 alkane of the said volatile mixture of alkanes is between 0 and 20% relative to the total mass of the said volatile mixture of alkanes.

In one embodiment, the composition of the eye makeup according to the invention is characterized in that the mass percentage of at least one linear or branched C>10 alkane of the said volatile mixture of alkanes is between 10% and 30% relative to the total mass of the said volatile mixture of alkanes.

In one embodiment, the composition of the eye makeup according to the invention is characterized in that the mass percentage of at least one linear or branched C>10 alkane of the said volatile mixture of alkanes is between 30% and 50% relative to the total mass of the said volatile mixture of alkanes.

In one embodiment, the composition of the eye makeup according to the invention is characterized in that the mass percentage of at least one linear or branched C>10 alkane of the said volatile mixture of alkanes is between 40% and 60% relative to the total mass of the said volatile mixture of alkanes.

In one embodiment, the composition of the eye makeup according to the invention is characterized in that the mass percentage of at least one linear or branched C>10 alkane of the said volatile mixture of alkanes is between 50% and 70% relative to the total mass of the said volatile mixture of alkanes.

In one embodiment, the composition of the eye makeup according to the invention is characterized in that the mass percentage of at least one linear or branched C>10 alkane of the said volatile mixture of alkanes is between 60% and 80% relative to the total mass of the said volatile mixture of alkanes.

In one embodiment, the composition of the eye makeup according to the invention is characterized in that the mass percentage of at least one linear or branched C>10 alkane of the said volatile mixture of alkanes is between 0% and 10% relative to the total mass of the said volatile mixture of alkanes.

In one embodiment, the composition of the eye makeup according to the invention is characterized in that the mass percentage of at least one linear or branched C>10 alkane of the said volatile mixture of alkanes is between 10% and 20% relative to the total mass of the said volatile mixture of alkanes.

In one embodiment, the composition of the eye makeup according to the invention is characterized in that the mass percentage of at least one linear or branched C>10 alkane of the said volatile mixture of alkanes is between 20% and 30% relative to the total mass of the said volatile mixture of alkanes.

In one embodiment, the composition of the eye makeup according to the invention is characterized in that the mass percentage of at least one linear or branched C>10 alkane of the said volatile mixture of alkanes is between 40% and 50% relative to the total mass of the said volatile mixture of alkanes.

In one embodiment, the composition of the eye makeup according to the invention is characterized in that the mass percentage of at least one linear or branched C>10 alkane of the said volatile mixture of alkanes is between 60% and 70% relative to the total mass of the said volatile mixture of alkanes.

In one embodiment, the composition of the eye makeup according to the invention is characterized in that the mass percentage of at least one linear or branched C>10 alkane of the said volatile mixture of alkanes is between 70% and 80% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is between 0 and 80% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is between 0 and 50% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is between 0 and 30% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is at least 10% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is at least 30% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is between 10% and 80% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is between 30% and 80% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is between 30% and 60% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is between 50% and 80% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is between 30% and 50% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is between 40% and 60% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is between 50% and 70% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is between 60% and 80% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is between 0% and 10% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is between 10% and 20% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is between 20% and 30% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is between 40% and 50% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is between 50% and 60% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is between 60% and 70% relative to the total mass of the said volatile mixture of alkanes.

In one embodiment, the composition of the eye makeup according to the invention is characterized in that the mass percentage of n-dodecane (CAS 112-40-3) of at least one linear or branched C>10 alkane of the said volatile mixture of alkanes is between 70% and 80% relative to the total mass of the said volatile mixture of alkanes.

The invention also relates to an eye makeup composition comprising a volatile mixture of alkanes as previously defined, characterized in that the volatile mixture of alkanes comprises:
20 to 100% trimethylheptane (CAS 4032-93-3).
0 to 80% n-dodecane (CAS 112-40-3).

In one embodiment, the composition of the eye makeup according to the invention is characterized in that the volatile mixture of alkanes consists of:
20 to 100% trimethylheptane (CAS 4032-93-3).
0 to 80% n-dodecane (CAS 112-40-3).

In one embodiment, the composition of the eye makeup according to the invention is characterized in that the volatile mixture of alkanes comprises:
20 to 70% trimethylheptane (CAS 4032-93-3)
30 to 80% n-dodecane (CAS 112-40-3).

In one embodiment, the composition of the eye makeup according to the invention is characterized in that the volatile mixture of alkanes consists of:
20 to 70% trimethylheptane (CAS 4032-93-3)
30 to 80% n-dodecane (CAS 112-40-3).

In one embodiment, the composition of the eye makeup according to the invention is characterized in that the volatile mixture of alkanes comprises:
50 to 100% trimethylheptane (CAS 4032-93-3)
0 to 50% n-dodecane (CAS 112-40-3).

In one embodiment, the composition of the eye makeup according to the invention is characterized in that the volatile mixture of alkanes consists of:
50 to 70% trimethylheptane (CAS 4032-93-3)
0 to 50% n-dodecane (CAS 112-40-3).

In one embodiment, the composition of the eye makeup according to the invention is characterized in that the volatile mixture of alkanes comprises:
20 to 100% of 2,3,6-trimethylheptane (CAS 4032-93-3)
0 to 80% n-dodecane (CAS 112-40-3).

In one embodiment, the composition of the eye makeup according to the invention is characterized in that the volatile mixture of alkanes consists of:
20 to 100% of 2,3,6-trimethylheptane (CAS 4032-93-3)
0 to 80% n-dodecane (CAS 112-40-3).

In one embodiment, the composition of the eye makeup according to the invention is characterized in that the volatile mixture of alkanes comprises:
20 to 70% of 2,3,6-trimethylheptane (CAS 4032-93-3)
30 to 80% n-dodecane (CAS 112-40-3).

In one embodiment, the composition of the eye makeup according to the invention is characterized in that the volatile mixture of alkanes consists of:
20 to 70% of 2,3,6-trimethylheptane (CAS 4032-93-3)
30 to 80% n-dodecane (CAS 112-40-3).

In one embodiment, the composition of the eye makeup according to the invention is characterized in that the volatile mixture of alkanes consists of:
30% 2,3,6-trimethylheptane (CAS 4032-93-3)
70% n-dodecane (CAS 112-40-3).

In one embodiment, the composition of the eye makeup according to the invention is characterized in that the volatile mixture of alkanes consists of:
35% 2,3,6-trimethylheptane (CAS 4032-93-3)
65% n-dodecane (CAS 112-40-3)

In one embodiment, the composition of the eye makeup according to the invention is characterized in that the volatile mixture of alkanes consists of:
40% 2,3,6-trimethylheptane (CAS 4032-93-3)
60% n-dodecane (CAS 112-40-3).

In one embodiment, the composition of the eye makeup according to the invention is characterized in that the volatile mixture of alkanes consists of:
45% 2,3,6-trimethylheptane (CAS 4032-93-3)
55% n-dodecane (CAS 112-40-3).

In one embodiment, the composition of the eye makeup according to the invention is characterized in that the volatile mixture of alkanes consists of:
60% 2,3,6-trimethylheptane (CAS 4032-93-3)
40% n-dodecane (CAS 112-40-3).

In one embodiment, the composition of the eye makeup according to the invention is characterized in that the volatile mixture of alkanes consists of:
70% 2,3,6-trimethylheptane (CAS 4032-93-3)
30% n-dodecane (CAS 112-40-3).

In one embodiment, the composition of the eye makeup according to the invention is characterized in that the volatile mixture of alkanes consists of:
90% 2,3,6-trimethylheptane (CAS 4032-93-3)
10% n-dodecane (CAS 112-40-3).

In one embodiment, the composition of the eye makeup according to the invention is characterized in that the volatile mixture of alkanes consists of:
100% 2,3,6-trimethylheptane (CAS 4032-93-3).

In one embodiment, the composition of the eye makeup according to the invention is characterized in that the volatile mixture of alkanes comprises:
5 to 15% trimethylhexane
20 to 95% trimethylheptane
0 to 75% n-dodecane (CAS 112-40-3).

In one embodiment, the composition of the eye makeup according to the invention is characterized in that the volatile mixture of alkanes consists of:
5 to 15% trimethylhexane
20 to 95% trimethylheptane
0 to 75% n-dodecane (CAS 112-40-3).

In one embodiment, the composition of the eye makeup according to the invention is characterized in that the volatile mixture of alkanes comprises:
5 to 10% of 2,2,4-trimethylhexane
20 to 60% of 2,3,6-trimethylheptane
0 to 75% n-dodecane (CAS 112-40-3).

In one embodiment, the composition of the eye makeup according to the invention is characterized in that the volatile mixture of alkanes consists of:
5 to 10% of 2,2,4-trimethylhexane
20 to 60% of 2,3,6-trimethylheptane
0 to 75% n-dodecane (CAS 112-40-3).

In one embodiment, the composition of the eye makeup according to the invention is characterized in that the volatile mixture of alkanes comprises:
5 to 15% of 2,2,4-trimethylhexane (CAS 16747-26-5)
20 to 95% of 2,3,6-trimethylheptane (CAS 4032-93-3)
0 to 75% n-dodecane (CAS 112-40-3).

In one embodiment, the composition of the eye makeup according to the invention is characterized in that the volatile mixture of alkanes consists of:
5 to 15% of 2,2,4-trimethylhexane (CAS 16747-26-5)
20 to 95% of 2,3,6-trimethylheptane (CAS 4032-93-3)
0 to 75% n-dodecane (CAS 112-40-3).

In one embodiment, the composition of the eye makeup according to the invention is characterized in that the volatile mixture of alkanes comprises:
5 to 10% of 2,2,4-trimethylhexane (CAS 16747-26-5)
20 to 60% of 2,3,6-trimethylheptane (CAS 4032-93-3)
0 to 75% n-dodecane (CAS 112-40-3).

In one embodiment, the composition of the eye makeup according to the invention is characterized in that the volatile mixture of alkanes consists of:
5 to 10% of 2,2,4-trimethylhexane (CAS 16747-26-5)
20 to 60% of 2,3,6-trimethylheptane (CAS 4032-93-3)
0 to 75% n-dodecane (CAS 112-40-3).

In one embodiment, the composition of the eye makeup according to the invention is characterized in that the volatile mixture of alkanes consists of:
5% 2,2,4-trimethylhexane (CAS 16747-26-5)
20% 2,3,6-trimethylheptane (CAS 4032-93-3)
75% n-dodecane (CAS 112-40-3).

In one embodiment, the composition of the eye makeup according to the invention is characterized in that the volatile mixture of alkanes consists of:
5% 2,2,4-trimethylhexane (CAS 16747-26-5)
30% 2,3,6-trimethylheptane (CAS 4032-93-3)
65% n-dodecane (CAS 112-40-3).

In one embodiment, the composition of the eye makeup according to the invention is characterized in that the volatile mixture of alkanes consists of:
5% 2,2,4-trimethylhexane (CAS 16747-26-5)
35% 2,3,6-trimethylheptane (CAS 4032-93-3)
60% n-dodecane (CAS 112-40-3).

In one embodiment, the composition of the eye makeup according to the invention is characterized in that the volatile mixture of alkanes consists of:
5% 2,2,4-trimethylhexane (CAS 16747-26-5)
45% 2,3,6-trimethylheptane (CAS 4032-93-3)
50% n-dodecane (CAS 112-40-3).

In one embodiment, the composition of the eye makeup according to the invention is characterized in that the volatile mixture of alkanes consists of:
10% 2,2,4-trimethylhexane (CAS 16747-26-5)
20% 2,3,6-trimethylheptane (CAS 4032-93-3)
70% n-dodecane (CAS 112-40-3).

In one embodiment, the composition of the eye makeup according to the invention is characterized in that the volatile mixture of alkanes consists of:
10% 2,2,4-trimethylhexane (CAS 16747-26-5)
30% 2,3,6-trimethylheptane (CAS 4032-93-3)
60% n-dodecane (CAS 112-40-3).

In one embodiment, the composition of the eye makeup according to the invention is characterized in that the volatile mixture of alkanes consists of:
10% 2,2,4-trimethylhexane (CAS 16747-26-5)
35% 2,3,6-trimethylheptane (CAS 4032-93-3)
55% n-dodecane (CAS 112-40-3).

In one embodiment, the composition of the eye makeup according to the invention is characterized in that the volatile mixture of alkanes consists of:
15% 2,2,4-trimethylhexane (CAS 16747-26-5)
20% 2,3,6-trimethylheptane (CAS 4032-93-3)
65% n-dodecane (CAS 112-40-3).

In one embodiment, the composition of the eye makeup according to the invention is characterized in that the volatile mixture of alkanes consists of:
15% 2,2,4-trimethylhexane (CAS 16747-26-5)
25% 2,3,6-trimethylheptane (CAS 4032-93-3)
60% n-dodecane (CAS 112-40-3).

In one embodiment, the composition of the eye makeup according to the invention is characterized in that the volatile mixture of alkanes consists of:
15% 2,2,4-trimethylhexane (CAS 16747-26-5)
30% 2,3,6-trimethylheptane (CAS 4032-93-3)
55% n-dodecane (CAS 112-40-3).

In one embodiment, the composition of the eye makeup according to the invention as defined above is characterized in that the volatile mixture of alkanes comprises:
a) from 70 to 99% by mass of the volatile mixture of alkanes composed of a fraction A and a fraction B as defined above
b) from 1 to 30% by mass of a C fraction containing at least one linear C≤10 alkane.

In one embodiment, the composition of the eye makeup according to the invention is characterized in that the volatile mixture of alkanes comprises at least one linear C≤10 alkane selected from the group consisting of n-octane, n-nonane and n-decane.

In one embodiment, the composition of the eye makeup according to the invention is characterized in that at least one linear C≤10 alkane present in the volatile mixture of alkanes is n-decane.

In one embodiment, the composition of the eye makeup according to the invention is characterized in that the mass percentage of at least one linear C≤10 alkane is at least 1% relative to the total mass of the said volatile mixture of alkanes.

In one embodiment, the composition of the eye makeup according to the invention is characterized in that the mass percentage of at least one linear C≤10 alkane is between 1 and 20% relative to the total mass of the said volatile mixture of alkanes.

In one embodiment, the composition of the eye makeup according to the invention is characterized in that the mass percentage of at least one linear C≤10 alkane is between 10 and 30% relative to the total mass of the said volatile mixture of alkanes.

In one embodiment, the composition of the eye makeup according to the invention is characterized in that the mass percentage of at least one linear C≤10 alkane is between 5 and 15% relative to the total mass of the said volatile mixture of alkanes.

In one embodiment, the composition of the eye makeup according to the invention is characterized in that the mass percentage of at least one linear C≤10 alkane is between 10 and 20% relative to the total mass of the said volatile mixture of alkanes.

In one embodiment, the composition of the eye makeup according to the invention is characterized in that the mass percentage of at least one linear C≤10 alkane is between 20 and 30% relative to the total mass of the said volatile mixture of alkanes.

In one embodiment, the composition of the eye makeup according to the invention is characterized in that the volatile mixture of alkanes comprising a fraction A, a fraction B and a fraction C as defined above has a flash point below 55° C. according to the ASTM D93 standard.

In one embodiment, the composition of the eye makeup according to the invention is characterized in that the volatile mixture of alkanes comprising a fraction A, a fraction B and a fraction C as defined above has a flash point of between 45 and 54° C.

In one embodiment, the composition of the eye makeup according to the invention is characterized in that the volatile mixture of alkanes comprising a fraction A, a fraction B and a fraction C as defined above has a flash point of between 45 and 50° C.

In one embodiment, the composition of the eye makeup according to the invention is characterized in that the volatile mixture of alkanes comprising a fraction A, a fraction B and a fraction C as defined above has a flash point of between 50 and 54° C.

In one embodiment, the volatile mixture of alkanes comprising a fraction A, a fraction B and a fraction C as defined above has a percentage greater than or equal to 50% in carbon 14 (¹⁴C) according to one of the following methods ASTM D6866, EM 16640 or EN 16785-1.

In one embodiment, the volatile mixture of alkanes comprising a fraction A, a fraction B and a fraction C as defined above has a percentage greater than or equal to 60% in carbon 14 (¹⁴C) according to one of the following methods ASTM D6866, EM 16640 or EN 16785-1.

In one embodiment, the volatile mixture of alkanes comprising a fraction A, a fraction B and a fraction C as defined above has a percentage greater than or equal to 75% in carbon 14 (¹⁴C) according to one of the following methods ASTM D6866, EM 16640 or EN 16785-1.

In one embodiment, the volatile mixture of alkanes comprising a fraction A, a fraction B and a fraction C as defined above has a percentage greater than or equal to 85% in carbon 14 (¹⁴C) according to one of the following methods ASTM D6866, EM 16640 or EN 16785-1.

In one embodiment, the volatile mixture of alkanes comprising a fraction A, a fraction B and a fraction C as defined above has a percentage greater than or equal to 90% in carbon 14 (¹⁴C) according to one of the following methods ASTM D6866, EM 16640 or EN 16785-1.

In the context of the present application, the composition of the eye makeup is characterized in that it comprises at least one non-volatile oil.

For the purposes of the present invention, the term “non-volatile oil” means an oil having a flash point greater than 110° C. measured according to the ATSM D93 standard.

These oils can be of vegetable, mineral or synthetic origin.

These non-volatile oils can be hydrocarbon, silicone, fluorinated or fluorosilicone oils.

For the purposes of the present invention, the term “hydrocarbon oil” means an oil essentially consisting of carbon atoms, hydrogen atoms and optionally oxygen and/or nitrogen atoms.

Among these hydrocarbon oils, mention may be made of linear or branched alkanes of mineral or synthetic origin such as linear or branched alkanes of 17 to 40 carbon atoms, (poly)esters and (poly)ethers, and in particular (poly)esters of C₂-C₂₄ acids (preferably C₆-C₂₀), triglycerides of C₆-C₂₀ fatty acids, vegetable oils, dialkyl carbonates, branched and/or unsaturated fatty acids, branched and/or unsaturated fatty alcohols.

Concerning non-volatile silicone oils, they are notably chosen from the group comprising phenylated silicone oils, non-volatile polydimethylsiloxanes, or derivatives of non-volatile polydimethylsiloxanes and their mixtures.

In a particularly preferred embodiment of the invention, the composition of the eye makeup according to the invention is characterized in that the non-volatile oil is chosen from the group consisting of non-volatile linear or branched alkanes.

Preferably, the composition of the eye makeup according to the invention is characterized in that the non-volatile oil is chosen from the group consisting of non-volatile linear or branched alkanes.

Preferably, the composition of the eye makeup according to the invention is characterized in that the non-volatile linear or branched alkanes are chosen from the group consisting of linear or branched alkanes of 17 to 40 carbon atoms.

The non-volatile oils used in this invention are of plant origin.

In a preferred embodiment, the composition of the eye makeup according to the invention is characterized in that the non-volatile oil is a vegetable hydrocarbon oil.

The vegetable hydrocarbon oils are notably chosen from the group comprising wheat germ, sunflower, grape seed, sesame, coconut, apricot, castor, corn, avocado, soybean, shea, olive, sweet almond oil, cotton, palm, macadamia, rapeseed, jojoba, hazelnut, poppy, alfalfa, pumpkin, blackcurrant, squash, evening primrose, barley oils, and mixtures thereof.

In one embodiment, the composition of the eye makeup according to the invention is characterized in that the mass percentage of the non-volatile oil is at least 5% relative to the total mass of the composition.

In one embodiment, the composition of the eye makeup according to the invention is characterized in that the mass percentage of the non-volatile oil is at least 10% relative to the total mass of the composition.

In one embodiment, the composition of the eye makeup according to the invention is characterized in that the mass percentage of the non-volatile oil is between 5 and 25% relative to the total mass of the composition.

In one embodiment, the composition of the eye makeup according to the invention is characterized in that the mass percentage of the non-volatile oil is between 5 and 15% relative to the total mass of the composition.

In one embodiment, the composition of the eye makeup according to the invention is characterized in that the mass percentage of the non-volatile oil is between 10 and 25% relative to the total mass of the composition.

For the purposes of the present invention, the term “glossy oil” means an oil having intrinsic gloss properties. “Glossy oils” are frequently used in cosmetics to ensure a “glossy effect” when applying cosmetic compositions to keratin materials, for example.

In the context of the present application, the composition of the eye makeup is characterized in that it comprises at least one glossy oil.

In one embodiment, the composition of the eye makeup according to the invention is characterized in that the glossy oil is a non-volatile oil.

In one embodiment, the composition of the eye makeup according to the invention is characterized in that the glossy oil is chosen from the group comprising:
polymers such as: polybutylenes, in particular POLYBUTENE® marketed or manufactured by the company ATAMAN, hydrogenated polyisobutylenes, polydecenes and hydrogenated polydecenes, polyfarnesenes, vinylpyrrolidone copolymers, in particular the vinylpyrrolidone/eicosene copolymer, ANTARON V 220® marketed or manufactured by the ISP company.
esters such as esters of linear fatty acids having a total number of carbons greater than 30, aromatic esters, esters of fatty alcohols or branched fatty acids having from 20 to 30 carbon atoms.
ester copolymers such as: dimer dilinoleyl dimer dilinoleate (LUSPLAN DD-DA5® and LUSPLAN DD-DA7®), dilinoleic acid/butanediol copolymer (VISCOPLAST 14436H®), dilinoleic acid/propanediol copolymer and their mixtures.

Preferably, the glossy oil used in the context of the present invention is of plant origin.

In one embodiment, the composition of the eye makeup according to the invention is characterized in that the mass percentage of the glossy oil is at least 2% relative to the total mass of the composition.

In one embodiment, the composition of the eye makeup according to the invention is characterized in that the mass percentage of the glossy oil is at least 5% relative to the total mass of the composition.

In one embodiment, the composition of the eye makeup according to the invention is characterized in that the mass percentage of the glossy oil is between 2 and 20% relative to the total mass of the composition.

In one embodiment, the composition of the eye makeup according to the invention is characterized in that the mass percentage of the glossy oil is between 5 and 15% relative to the total mass of the composition.

In one embodiment, the composition of the eye makeup according to the invention is characterized in that the mass percentage of the glossy oil is between 10 and 20% relative to the total mass of the composition.

In one embodiment, the composition of the eye makeup according to the invention is characterized in that the mass percentage of the glossy oil is between 2 and 10% relative to the total mass of the composition.

In one embodiment, the composition of the eye makeup according to the invention is characterized in that the mass percentage of the glossy oil is between 5 and 10% relative to the total mass of the composition.

These ingredients play an important role in formulating composition of the eye makeups as they help balance the formulation in terms of creaminess during use.

For the purposes of the present invention, the term “pasty fatty substance” means a lipophilic fatty compound with a reversible solid/liquid state change, having at room temperature (25° C.) a liquid fraction and a solid fraction.

In other words, the melting temperature of the pasty fat is less than or equal to 25° C.

In the context of the present application, the composition of the eye makeup is characterized in that it further comprises at least one pasty fatty substance.

In one embodiment, the composition of the eye makeup according to the invention is characterized in that the pasty fatty substance is chosen from the group comprising synthetic pasty fatty substances, animal pasty fatty substances, vegetable pasty fatty substances and their mixtures.

In one embodiment, the composition of the eye makeup according to the invention is characterized in that the synthetic pasty fatty substance is chosen from the group comprising: polyol ethers, petroleum jelly, vinyl polymers, pentaerythritol esters, polyesters and their mixtures.

In one embodiment, the composition of the eye makeup according to the invention is characterized in that the animal pasty fatty substance is lanolin.

Preferably, the composition of the eye makeup according to the invention is characterized in that the pasty fatty substance is a vegetable pasty fatty substance.

In one embodiment, the composition of the eye makeup according to the invention is characterized in that the vegetable pasty fatty substance is chosen from the group comprising: vegetable butters (avocado butter, shea butter), fatty acid triglycerides and their derivatives, and their mixtures.

In one embodiment, the composition of the eye makeup according to the invention is characterized in that the mass percentage of the pasty fatty substance is between 0 and 20% relative to the total mass of the composition.

In one embodiment, the composition of the eye makeup according to the invention is characterized in that the mass percentage of the pasty fatty substance is between 0 and 15% relative to the total mass of the composition.

In one embodiment, the composition of the eye makeup according to the invention is characterized in that the mass percentage of the pasty fatty substance is between 0 and 10% relative to the total mass of the composition.

In one embodiment, the composition of the eye makeup according to the invention is characterized in that the mass percentage of the pasty fatty substance is between 0 and 5% relative to the total mass of the composition.

For the purposes of the present invention, the term gelling agent means compounds which constitute excellent texturizing agents for balancing formulations in terms of smoothness and stickiness.

Furthermore, certain glossy oils such as VISCOPLAST 14436H®, and compounds marketed under the names LUSPLAN DD-DA5® and LUSPLAN DD-DA7® can also be used as gelling agents.

In one embodiment, the composition of the eye makeup according to the invention is characterized in that it comprises at least one gelling agent.

In one embodiment, the composition of the eye makeup according to the invention is characterized in that the gelling agent is chosen from the group comprising: organic gelling agents, mineral gelling agents, polymeric gelling agents, and mixtures thereof.

Preferably, the composition of the eye makeup according to the invention is characterized in that the gelling agent is of plant origin.

In one embodiment, the composition of the eye makeup according to the invention is characterized in that the organic gelling agent is chosen from the group comprising: ethylcellulose and its derivatives, resins derived from rosin, dextrin esters, fatty acid esters, and mixtures thereof.

In one embodiment, the composition of the eye makeup according to the invention is characterized in that the mineral gelling agent is chosen from the group comprising: hectorite and its derivatives, pyrogenic silica and its derivatives, and mixtures thereof.

In one embodiment, the composition of the eye makeup according to the invention is characterized in that the polymeric gelling agent is selected from the group comprising: organopolysiloxanes and their derivatives, dimethicone copolymers and their derivatives, polystyrene/polyisoprene copolymers, polystyrene/polybutadiene copolymers, polystyrene/copoly(ethylene-propylene) copolymers, polystyrene/copoly(ethylene-butylene) copolymers, mixtures of copolymers in isododecane such as ethylene/propylene copolymer or butylene/ethylene/styrene copolymer, polyesters and their derivatives, polyamide resins, and mixtures thereof.

In one embodiment, the composition of the eye makeup according to the invention is characterized in that the gelling agent is dextrin palmitate or one of its derivatives.

In one embodiment, the composition of the eye makeup according to the invention is characterized in that the gelling agent is a modified hectorite of the BENTONE GEL ISD V® type or one of its equivalents.

In one embodiment, the composition of the eye makeup according to the invention is characterized in that the gelling agent is a modified hectorite of the VEGELIGHT BENTONE SILK SB® type or one of its equivalents.

In one embodiment, the composition of the eye makeup according to the invention is characterized in that the gelling agent is a modified hectorite of the disteardimonium hectorite type associated with triethyl citrate and a volatile mixture of alkanes defined according to the present invention.

In one embodiment, the composition of the eye makeup according to the invention is characterized in that the gelling agent is a mixture of copolymer in isododecane such as ethylene/propylene copolymer.

In one embodiment, the composition of the eye makeup according to the invention is characterized in that the gelling agent is the combination of a dimethicone polymer and a mixture of alkanes marketed under the name VEGELIGHT SILK SI 118® by BIOSYNTHIS, or one of its equivalents.

In one embodiment, the composition of the eye makeup according to the invention is characterized in that the mass percentage of the gelling agent is at least 5% relative to the total mass of the composition.

In one embodiment, the composition of the eye makeup according to the invention is characterized in that the mass percentage of the gelling agent is at least 15% relative to the total mass of the composition.

In one embodiment, the composition of the eye makeup according to the invention is characterized in that the mass percentage of the gelling agent is between 5 and 30% relative to the total mass of the composition.

In one embodiment, the composition of the eye makeup according to the invention is characterized in that the mass percentage of the gelling agent is between 5 and 20% relative to the total mass of the composition.

In one embodiment, the composition of the eye makeup according to the invention is characterized in that the mass percentage of the gelling agent is between 15 and 30% relative to the total mass of the composition.

In one embodiment, the composition of the eye makeup according to the invention is characterized in that the mass percentage of the gelling agent is between 5 and 15% relative to the total mass of the composition.

In one embodiment, the composition of the eye makeup according to the invention is characterized in that the mass percentage of the gelling agent is between 15 and 25% relative to the total mass of the composition.

In one embodiment, the composition of the eye makeup according to the invention is characterized in that the mass percentage of the gelling agent is between 20 and 30% relative to the total mass of the composition.

For the purposes of the present invention, the term film-forming polymer means a polymer capable of forming a macroscopically continuous film, in particular on the eyelashes or the skin within the framework of our invention.

In one embodiment, the composition of the eye makeup according to the invention is characterized in that it comprises at least one film-forming polymer.

In one embodiment, the composition of the eye makeup according to the invention is characterized in that the film-forming polymer is chosen from the group comprising: acrylic polymers, polyurethanes, polyesters, polyamides, silicone polymers.

In one embodiment, the composition of the eye makeup according to the invention is characterized in that the film-forming polymer is trimethylsilloxysilicate marketed under the name TMS 803.

In one embodiment, the composition of the eye makeup according to the invention is characterized in that the mass percentage of the film-forming polymer is at least 1% relative to the total mass of the composition.

In one embodiment, the composition of the eye makeup according to the invention is characterized in that the mass percentage of the film-forming polymer is at least 10% relative to the total mass of the composition.

In one embodiment, the composition of the eye makeup according to the invention is characterized in that the mass percentage of the film-forming polymer is between 1 and 25% relative to the total mass of the composition.

In one embodiment, the composition of the eye makeup according to the invention is characterized in that the mass percentage of the film-forming polymer is between 10 and 25% relative to the total mass of the composition.

In one embodiment, the composition of the eye makeup according to the invention is characterized in that the mass percentage of the film-forming polymer is between 1 and 10% relative to the total mass of the composition.

In one embodiment, the composition of the eye makeup according to the invention is characterized in that the mass percentage of the film-forming polymer is between 10 and 20% relative to the total mass of the composition.

Waxes play an important role in eye makeup design because they influence certain essential criteria such as the texture, appearance and/or strength of the product.

For the purposes of this application, “wax” means a lipophilic compound having a reversible solid/liquid state change. In other words, the compound is solid at room temperature (at 25° C.) and has a melting point greater than or equal to 30° C.

Preferably, for the purposes of the present invention, the waxes used have a melting point greater than or equal to 50° C.

In one embodiment, the composition of the eye makeup according to the invention is characterized in that it comprises at least one wax.

In one embodiment, the composition of the eye makeup according to the invention is characterized in that the wax is a synthetic wax, a mineral wax, a vegetable wax or an animal wax.

In one embodiment, the composition of the eye makeup according to the invention is characterized in that the wax is a mineral wax or a synthetic wax chosen from the group comprising: microcrystalline waxes, paraffins, ozokerite, polyethylene waxes, silicone waxes, fluorinated waxes.

By “vegetable wax” is meant a composition comprising at least one partially or completely hydrogenated oil. These vegetable waxes may comprise esters of fatty acids and fatty alcohols, and may further comprise fatty acids, free fatty alcohols, long chain linear alkanes and/or unsaponifiables.

Preferably, within the meaning of the present invention, the waxes used are vegetable waxes.

In one embodiment, the composition of the eye makeup according to the invention is characterized in that the wax is a vegetable or animal wax chosen from the group comprising: lanolin wax, rice bran wax, carnauba wax, candelilla wax, berry wax, beeswax, sunflower wax, mimosa wax, jojoba wax, castor wax, olive wax.

In one embodiment, the composition of the eye makeup according to the invention is characterized in that the mass percentage of the wax is at least 2% relative to the total mass of the composition.

In one embodiment, the composition of the eye makeup according to the invention is characterized in that the mass percentage of the wax is at least 5% relative to the total mass of the composition.

In one embodiment, the composition of the eye makeup according to the invention is characterized in that the mass percentage of the wax is between 2 and 20% relative to the total mass of the composition.

In one embodiment, the composition of the eye makeup according to the invention is characterized in that the mass percentage of the wax is between 5 and 15% relative to the total mass of the composition.

In one embodiment, the composition of the eye makeup according to the invention is characterized in that the mass percentage of the wax is between 2 and 10% relative to the total mass of the composition.

In one embodiment, the composition of the eye makeup according to the invention is characterized in that the mass percentage of the wax is between 5 and 10% relative to the total mass of the composition.

In one embodiment, the composition of the eye makeup according to the invention is characterized in that it further comprises at least one antioxidant.

In one embodiment, the composition of the eye makeup according to the invention is characterized in that the antioxidant is chosen from the group comprising: phenolic antioxidants of the BHA, BHT, DBPC, gallate type, so-called natural antioxidants such as dl-alpha-tocopherol or its derivatives, plant extracts, dibasic lipopeptides such as lysine or arginine, and mixtures thereof.

In one embodiment, the composition of the eye makeup according to the invention is characterized in that the mass percentage of the antioxidant is at least 0.01% relative to the total mass of the composition.

In one embodiment, the composition of the eye makeup according to the invention is characterized in that the mass percentage of the antioxidant is at least 0.1% relative to the total mass of the composition.

In one embodiment, the composition of the eye makeup according to the invention is characterized in that the mass percentage of the antioxidant is between 0.01% and 2% relative to the total mass of the composition.

In one embodiment, the composition of the eye makeup according to the invention is characterized in that the mass percentage of the antioxidant is between 0.01% and 1% relative to the total mass of the composition.

In one embodiment, the composition of the eye makeup according to the invention is characterized in that the mass percentage of the antioxidant is between 0.1% and 1% relative to the total mass of the composition.

In one embodiment, the composition of the eye makeup according to the invention is characterized in that the mass percentage of the antioxidant is between 0.01% and 0.1% relative to the total mass of the composition.

This part is essential to obtain the coloring. The pigments used are of synthetic, mineral or vegetable origin. Their dosage is also subtle.

In one embodiment, the composition of the eye makeup according to the invention is characterized in that it comprises at least one pigmentary part.

In one embodiment, the composition of the eye makeup according to the invention is characterized in that the pigmentary part is chosen from the group comprising: water-soluble dyes, fat-soluble dyes, pigments, mother-of-pearl, glitter and mixtures thereof.

In one embodiment, the composition of the eye makeup according to the invention is characterized in that the pigmentary part comprises a pigment chosen from the group comprising: organic pigments such as fixed pigments or precipitated pigments, mineral pigments such as titanium dioxide or iron oxides, sodium aluminosilicate thiosulfates, chromium oxides, bismuth oxychloride, copper phthalocyanine, metal-free phthalocyanine, chlorinated copper phthalocyanine.

Thus, the titanium oxide rate determines the coverage and opacity of the film.

The use of lacquers or precipitated pigments determines the color and chrominance.

For illustration purposes, other types of pigments can be used such as titanium mica, borosilicates and their derivatives, mica, silica.

Mineral pigments are used to nuance the hues while compounds such as bismuth oxychloride can be used to impart particular effects (metallic, shimmering effects, etc.).

In one embodiment, the composition of the eye makeup according to the invention is characterized in that the pigmentary part comprises a synthetic dye chosen from the group comprising:
CI77499, CI77267, CI77268, CI77499 CI77266, CI50420, CI27755, CI28440, CI20470 and mixtures thereof.

In one embodiment, the composition of the eye makeup according to the invention is characterized in that the pigmentary part comprises a dye of plant origin chosen from fruit and/or plant extracts containing dyes.

In one embodiment, the composition of the eye makeup according to the invention is characterized in that the mass percentage of the pigmentary part is at least 0.01% relative to the total mass of the composition.

In one embodiment, the composition of the eye makeup according to the invention is characterized in that the mass percentage of the pigmentary part is at least 1% relative to the total mass of the composition.

In one embodiment, the composition of the eye makeup according to the invention is characterized in that the mass percentage of the pigmentary part is at least 5% relative to the total mass of the composition.

In one embodiment, the composition of the eye makeup according to the invention is characterized in that the mass percentage of pigmentary part is between 0.01% and 20% relative to the total mass of the composition.

In one embodiment, the composition of the eye makeup according to the invention is characterized in that the mass percentage of pigmentary part is between 1% and 20% relative to the total mass of the composition.

In one embodiment, the composition of the eye makeup according to the invention is characterized in that the mass percentage of pigmentary part is between 5% and 20% relative to the total mass of the composition.

In one embodiment, the composition of the eye makeup according to the invention is characterized in that the mass percentage of pigmentary part is between 1% and 15% relative to the total mass of the composition.

In one embodiment, the composition of the eye makeup according to the invention is characterized in that the mass percentage of pigmentary part is between 5% and 15% relative to the total mass of the composition.

In one embodiment, the composition of the eye makeup according to the invention is characterized in that the mass percentage of pigmentary part is between 0.01% and 5% relative to the total mass of the composition.

In one embodiment, the composition of the eye makeup according to the invention is characterized in that the mass percentage of pigmentary part is between 5% and 10% relative to the total mass of the composition.

In one embodiment, the composition of the eye makeup according to the invention is characterized in that the mass percentage of pigmentary part is between 10% and 15% relative to the total mass of the composition.

In one embodiment, the composition of the eye makeup according to the invention is characterized in that it has an index of naturalness greater than or equal to 80% carbon 14 (¹⁴C) determined according to one of the following standards ASTM D6866, EN 16640 or EN 16785-1.

In one embodiment, the composition of the eye makeup according to the invention is characterized in that it has an index of naturalness greater than or equal to 85% carbon 14 (¹⁴C) determined according to one of the following standards ASTM D6866, EN 16640 or EN 16785-1.

In one embodiment, the composition of the eye makeup according to the invention is characterized in that it has an index of naturalness greater than or equal to 90% carbon 14 (¹⁴C) determined according to one of the following standards ASTM D6866, EN 16640 or EN 16785-1.

In one embodiment, the composition of the eye makeup according to the invention is characterized in that it has an index of naturalness greater than or equal to 95% carbon 14 (¹⁴C) determined according to one of the following standards ASTM D6866, EN 16640 or EN 16785-1.

In one embodiment, the composition of the eye makeup according to the invention is characterized in that at least 50% by mass of its ingredients is of natural origin relative to the total mass of the composition.

In one embodiment, the composition of the eye makeup according to the invention is characterized in that at least 75% by mass of its ingredients is of natural origin relative to the total mass of the composition.

In one embodiment, the composition of the eye makeup according to the invention is characterized in that at least 90% by mass of its ingredients is of natural origin relative to the total mass of the composition.

In one embodiment, the composition of the eye makeup according to the invention is characterized in that 50% by mass of its ingredients is of natural origin relative to the total mass of the composition.

In one embodiment, the composition of the eye makeup according to the invention is characterized in that 75% by mass of its ingredients is of natural origin relative to the total mass of the composition.

In one embodiment, the composition of the eye makeup according to the invention is characterized in that 90% by mass of its ingredients is of natural origin relative to the total mass of the composition.

In one embodiment, the composition of the eye makeup according to the invention is characterized in that the drying time at 20° C. after application of the composition to the eyelashes is less than or equal to 5 min.

In one embodiment, the composition of the eye makeup according to the invention is characterized in that the drying time at 20° C. after application of the composition to the eyelashes is less than or equal to 4 min.

In one embodiment, the composition of the eye makeup according to the invention is characterized in that the drying time at 20° C. after application of the composition to the eyelashes is equal to 3 min and 30 s.

In one embodiment, the composition of the eye makeup according to the invention is characterized in that the drying time at 20° C. after application of the composition to the eyelashes is equal to 3 min.

The composition of the eye makeup according to the invention is characterized in that its color is bright and very intense.

The satisfactory properties in terms of color and intensity reflect a good distribution of pigments on the eyelashes for the composition of mascara.

In addition to its color properties, the mascara according to the present invention has a satisfactory eyelash separation effect as well as satisfactory eyelash staying power and curl.

When it comes to eyeliner, this good distribution of pigments results in a dark, intense makeup that highlights the eye contour.

The eyeliner obtained according to the present invention has a long staying power, does not dry out the skin and does not crumble.

In one embodiment, the composition of the eye makeup according to the invention is characterized in that it is in the form of a paste, a pencil, a felt-tip pen, a water-in-oil emulsion or an oily continuous phase.

In a preferred embodiment, the composition of the eye makeup according to the invention is characterized in that, it is in the form of a composition with a continuous oily phase.

In a preferred embodiment, the composition of the eye makeup according to the invention is characterized in that, it is in the form of a composition with an anhydrous oily continuous phase.

In a preferred embodiment, the composition of the eye makeup according to the invention is characterized in that, it is in the form of a composition with an anhydrous oily continuous phase.

For the purposes of the present invention, the term “composition with an anhydrous oily continuous phase” means a composition whose aqueous phase is less than or equal to 5% by mass relative to the total mass of the composition.

Furthermore, the composition of the eye makeup according to the invention can be manufactured by known methods, generally used in the cosmetic field.

Furthermore, the composition of the eye makeup according to the invention can be manufactured by known methods, generally used in the cosmetic field.

The invention also relates to a method of preparing an eye makeup composition according to the present invention comprising the following steps:
a) Production of a C10 Guerbet alcohol, 2-isopropyl-5-methylhexane-1-ol (CAS 2051-33-4) by a Guerbet synthesis from isoamyl alcohol (CAS 123-51-3).
b) The mixture of the product formed in step 1) with at least one bio-sourced alcohol in C>10 to form a mixture of bio-sourced alcohols.
c) Obtaining a volatile mixture of alkanes consisting of 2,3,6-trimethylheptane (CAS 4032-93-3) and a C alkane>10, by a dehydration reaction followed by a hydrogenation reaction of the mixture of bio-sourced alcohols formed in step 2).
d) The addition of none, one or more alkanes to this volatile mixture of alkanes.
e) The combination of this volatile mixture of alkanes with at least one compound chosen from non-volatile oils, glossy oils, pasty fatty substances, gelling agents, film-forming polymers, waxes, pigments, dyes, and mixtures thereof.

Even more specifically, the invention also relates to a method for preparing an eye makeup composition according to the present invention comprising the following steps:
a) Production of a C10 Guerbet alcohol, 2-isopropyl-5-methylhexane-1-ol (CAS 2051-33-4) by a Guerbet synthesis from isoamyl alcohol (CAS 123-51-3).
b) Mixing the product formed in step 1) with bio-based n-dodecanol to form a mixture of bio-based alcohols.
c) Obtaining a volatile mixture of alkanes consisting of 2,3,6-trimethylheptane (CAS 4032-93-3) and n-dodecane (CAS 112-40-3) by a dehydration reaction followed by a hydrogenation reaction of the mixture of biosourced alcohols formed in step 2).
d) The addition of none, one or more alkanes to this volatile mixture of alkanes.
e) The combination of this volatile mixture of alkanes with at least one compound chosen from non-volatile oils, glossy oils, pasty fatty substances, gelling agents, film-forming polymers, waxes, pigments, dyes, and mixtures thereof.

In one embodiment, the method for preparing an eye makeup composition, defined according to the present invention, is characterized in that step 1 of the method, namely the Guerbet synthesis, is carried out from raw materials of plant, bacterial or animal origin.

In one embodiment, the method for preparing an eye makeup composition, defined according to the present invention, is characterized in that step 1 of the method, namely the Guerbet synthesis, is carried out using a base chosen from the group consisting of: KOH, NaOH, NaOCH₃, KOCH₃.

In one embodiment, the method for preparing an eye makeup composition, defined according to the present invention, is characterized in that step 1 of the method, namely the Guerbet synthesis, is carried out using a catalyst chosen from the group comprising: copper derivatives such as PriCat Cu 50/8, at least one metal from group VIII, such as nickel, palladium, platinum, their derivatives and their mixtures.

In one embodiment, the process for preparing an eye makeup composition, defined according to the present invention, is characterized in that step 1 of the process, namely the Guerbet synthesis, is carried out at temperatures between 150 and 300° C.

In one embodiment, the process for preparing an eye makeup composition, defined according to the present invention, is characterized in that step 1 of the process, namely the Guerbet synthesis, is carried out at pressures between 1 and 15 bars.

In one embodiment, the method for preparing an eye makeup composition, defined according to the present invention, is characterized in that step 1 of the method, namely the Guerbet synthesis, is carried out for a period of between 10 and 24 hours.

In one embodiment, the method for preparing an eye makeup composition, defined according to the present invention, is characterized in that step 1 of the method, namely the Guerbet synthesis, makes it possible to obtain a mass percentage of branched C10 alcohol greater than or equal to 75%.

In one embodiment, the method for preparing an eye makeup composition, defined according to the present invention, is characterized in that step 1 of the method, namely the Guerbet synthesis, makes it possible to obtain a mass percentage of branched C10 alcohol greater than or equal to 90%.

In one embodiment, the method for preparing an eye makeup composition, defined according to the present invention, is characterized in that step 1 of the method further comprises a purification step by distillation to remove impurities.

In one embodiment, the method for preparing an eye makeup composition, defined according to the present invention, is characterized in that step 1 of the method, namely the Guerbet synthesis, makes it possible to obtain a branched C10 alcohol whose percentage of carbon 14 (¹⁴C) is greater than or equal to 75% of carbon 14 (¹⁴C) determined according to one of the following standards ASTM D6866, EN 16640 or EN 16785-1.

In one embodiment, the method for preparing an eye makeup composition, defined according to the present invention, is characterized in that step 1 of the method, namely the Guerbet synthesis, makes it possible to obtain a branched C10 alcohol whose percentage of carbon 14 (¹⁴C) is greater than or equal to 90% determined according to one of the following standards ASTM D6866, EN 16640 or EN 16785-1.

In one embodiment, the method for preparing an eye makeup composition, defined according to the present invention, is characterized in that step 1 of the method, namely the Guerbet synthesis, makes it possible to obtain a branched C10 alcohol whose percentage of carbon 14 (¹⁴C) is 100% determined according to one of the following standards: ASTM D6866, EN 16640 or EN 16785-1.

In one embodiment, the method for preparing an eye makeup composition, defined according to the present invention, is characterized in that in step 2 of the method, at least one bio-sourced C≥12 alcohol chosen from the group consisting of: n-dodecanol, n-tetradecanol, and mixtures thereof is added.

In one embodiment, the method for preparing an eye makeup composition, defined according to the present invention, is characterized in that step 3 of the method is composed of a dehydration step then a hydrogenation step.

In one embodiment, the method for preparing an eye makeup composition, defined according to the present invention, is characterized in that the dehydration step of step 3 of the method is carried out using a catalyst chosen from the group consisting of alumina and its derivatives.

In one embodiment, the method for preparing an eye makeup composition, defined according to the present invention, is characterized in that the dehydration step of step 3 of the method is carried out using a catalytic support chosen from the group consisting of silica wool and its derivatives.

In one embodiment, the process for preparing an eye makeup composition, defined according to the present invention, is characterized in that the dehydration step of step 3 of the process is carried out at temperatures between 200 and 380° C.

In one embodiment, the process for preparing an eye makeup composition, defined according to the present invention, is characterized in that the dehydration step of step 3 of the process is carried out under an inert atmosphere.

In one embodiment, the process for preparing an eye makeup composition, defined according to the present invention, is characterized in that the dehydration step of step 3 of the process makes it possible to obtain a conversion into branched C10 alkene greater than or equal to 95%.

In one embodiment, the method for preparing an eye makeup composition, defined according to the present invention, is characterized in that the dehydration step of step 3 of the method makes it possible to obtain a conversion into branched C10 alkene greater than or equal to 99%.

In one embodiment, the process for preparing an eye makeup composition, defined according to the present invention, is characterized in that the trimethylheptane obtained in the hydrogenation step of step 3 of the process is 2,3,6-trimethylheptane.

In one embodiment, the process for preparing an eye makeup composition, defined according to the present invention, is characterized in that the hydrogenation step of step 3 of the process is carried out using a catalyst comprising at least one metal from group VIII such as palladium, nickel, platinum, their derivatives and their mixtures.

In one embodiment, the process for preparing an eye makeup composition, defined according to the present invention, is characterized in that the hydrogenation step of step 3 of the process is carried out using a catalytic support chosen from the group consisting of silica wool and its derivatives.

In one embodiment, the process for preparing an eye makeup composition, defined according to the present invention, is characterized in that the hydrogenation step of step 3 of the process is carried out at temperatures between 100 and 250° C.

In one embodiment, the process for preparing an eye makeup composition, defined according to the present invention, is characterized in that the hydrogenation step of step 3 of the process is carried out under an inert atmosphere.

In one embodiment, the process for preparing an eye makeup composition, defined according to the present invention, is characterized in that the hydrogenation step of step 3 of the process is preceded by inerting with 15 bars of nitrogen in the reactor.

In one embodiment, the process for preparing an eye makeup composition, defined according to the present invention, is characterized in that the hydrogenation step of step 3 of the process is preceded by inerting with 5 bars of nitrogen three times in the reactor.

In one embodiment, the method for preparing an eye makeup composition, defined according to the present invention, is characterized in that the hydrogenation step of step 3 of the method is carried out by adding from 2 to 15 bars of hydrogen to the reactor.

In one embodiment, the method for preparing an eye makeup composition, defined according to the present invention, is characterized in that the hydrogenation step of step 3 of the method is carried out by adding 5 bars of hydrogen to the reactor.

In one embodiment, the method for preparing an eye makeup composition, defined according to the present invention, is characterized in that the hydrogenation step of step 3 of the method is carried out by adding 10 bars of hydrogen to the reactor.

In one embodiment, the method for preparing an eye makeup composition defined according to the present invention is characterized in that the hydrogenation step of step 3 of the method is carried out for a period of 3 to 12 hours.

In one embodiment, the process for preparing an eye makeup composition, defined according to the present invention, is characterized in that the hydrogenation step of step 3 of the process is carried out for a period of 5 hours.

In one embodiment, the process for preparing an eye makeup composition, defined according to the present invention, is characterized in that the hydrogenation step of step 3 of the process makes it possible to obtain a conversion into 2,3,6-trimethylheptane greater than or equal to 95%.

In one embodiment, the process for preparing an eye makeup composition, defined according to the present invention, is characterized in that the hydrogenation step of step 3 of the process makes it possible to obtain a conversion into 2,3,6-trimethylheptane greater than or equal to 99%.

In one embodiment, the method for preparing an eye makeup composition, defined according to the present invention, is characterized in that the method allows obtaining a volatile alkane mixture defined according to the present invention.

Surprisingly, the process for preparing an eye makeup composition, defined according to the present invention, is characterized in that the process allows the production of a deodorized or odorless volatile alkane mixture.

This organoleptic characteristic is conducive to its use in cosmetic compositions.

In one embodiment, the process for preparing an eye makeup composition, defined according to the present invention, is characterized in that step 4 of the process makes it possible to obtain a volatile alkane mixture defined according to the present invention.

In one embodiment, the method for preparing an eye makeup composition, defined according to the present invention, is characterized in that step 4 of the method makes it possible to obtain a volatile mixture of alkanes whose percentage of carbon 14 (¹⁴C) is greater than or equal to 75% of carbon 14 (¹⁴C) determined according to one of the following standards ASTM D6866, EN 16640 or EN 16785-1.

In one embodiment, the method for preparing an eye makeup composition, defined according to the present invention, is characterized in that step 4 of the method makes it possible to obtain a volatile mixture of alkanes whose percentage of carbon 14 (¹⁴C) is greater than or equal to 90% determined according to one of the following standards: ASTM D6866, EN 16640 or EN 16785-1.

In one embodiment, the method for preparing an eye makeup composition, defined according to the present invention, is characterized in that step 4 of the method makes it possible to obtain a volatile mixture of alkanes whose percentage of carbon 14 (¹⁴C) is 100% determined according to one of the following standards ASTM D6866, EN 16640 or EN 16785-1.

In one embodiment, the method for preparing an eye makeup composition, defined according to the present invention, is characterized in that step 5 of the method makes it possible to obtain an eye makeup composition comprising a volatile alkane mixture defined according to the present invention.

### EXAMPLES

The following examples are given to illustrate the invention. These examples being presented for illustration purposes only, the invention cannot in any case be limited to their purpose.

The percentages given in the tables below are mass percentages.

### Example 1: Protocol for Preparing Mascara Formulations

In order to prepare the reference mascara and those described according to the invention, phase 1 is weighed and heated to a maximum temperature of 35° C.

Separately, phase 2 is weighed into another container and then phase 2 is heated to a temperature of 60° C. Then, phase 2 is added to phase 1, as well as phase 3 in a second step.

Phase 4 is then added with vigorous magnetic stirring to obtain an emulsion.

Finally, the powders from phase 5 are added to the mixture.

### Example 2: Composition and Properties of a Reference Mascara

TABLE 1
Composition of a reference mascara

| | | | % by |
|---|---|---|---|
| PHASE | TRADE NAME | INCI | mass |
| 1 | ISODODECANE | ISODODECANE | 25.3 |
| | VEGELIGHT | COCONUT ALKANES (AND) | 10 |
| | BENTONE SILK | DISTEARDIMONIUM | |
| | SB | HECTORITE (AND) | |
| | | TRIETHYL CITRATE | |
| | TMS 803 | TRIMETHYLSILOXYSILICATE | 1 |
| | MI BLACK | ALUMINUM DIMYRISTATE | 14 |
| | | CI 77499 | |
| | MASSOCARE | PEG-30 | 1 |
| | PEG30 PHS | DIPOLYHYDROXYSTEARATE | |
| | OLIVE GEL SQE | DIMER DILINOLEYL DIMER | 5.0 |
| | | DILINOLEATE& | |
| | | OLIVE (OLEA EUROPAEA) | |
| | | OIL UNSAPONIFIABLES & | |
| | | DILINOLEIC | |
| | | ACID/BUTANEDIOL | |
| | | COPOLYMER | |
| 2 | ANTARON V216 | PVP/HEXADECENE | 3.0 |
| | (GANEX) | COPOLYMER | |
| | KAHLRESIN 6723 | SHOREA ROBUSTA RESIN & | 1.5 |
| | | OCTYLODECANOL | |
| | MYRITOL 318 | CAPRYLIC/CAPRIC | 1.8 |
| | | TRIGLYCERIDE | |
| | CEGESOFT HF 52 | HYDROGENATED | 10.0 |
| | | VEGETABLE OIL | |
| | BEESWAX | BEESWAX | 3.0 |
| | WHITE SP 422P | | |
| | CARNAUBA | COPERNICIA CERIFERA | 2.5 |
| | WAX SP 63P | (CARNAUBA) WAX | |
| | CRODABOND | HYDROGENATED CASTOR | 3.0 |
| | CSA LQ | OIL/SEBACIC ACID | |
| | | COPOLYMER | |
| | PARLEAM | HYDROGENATED | 2.0 |
| | | POLYISOBUTENE | |
| 3 | PHENOXETOL ® | PHENOXYETHANOL | 0.7 |
| | DERMOSOFT | CAPRYLYL GLYCOL | 0.5 |
| | OCTIOL | | |
| 4 | DEIONIZED | WATER | 10.0 |
| | WATER | | |
| | POTASSIUM | POTASSIUM SORBATE | 0.2 |
| | SORBATE | | |
| 5 | SILICA BEAD | SB 700 SILICA | 2 |
| | MIYOSYN | MICA (AND) ISOPROPYL | 2 |
| | MATTE-ALT | TITANIUM | |
| | | TRIISOSTEARATE | |
| | SENSOSEL 10 | CELLULOSE | 1 |
| | ESP ECOCEL 10 | CELLULOSE | 0.5 |

### Reference Mascara Drying Time

The drying time at 20° C. is measured after application to the eyelashes of a panel of users, by carrying out tests to verify drying using a non-woven tissue from t0 to tx, a contact test with a non-woven tissue being carried out every 30 seconds. The formulation is considered dry when there is no more stickiness.

The reference mascara obtained according to the protocol of example 1 has a drying time on the eyelashes equal to 2 min 50 sec.

This drying time is satisfactory for a mascara formulation.

### Application Characteristics of the Reference Mascara

To establish this study, the formulation of the reference mascara was tested by a panel of users.

Thus, according to this study, the reference mascara has a satisfactory appearance in the form of a smooth, homogeneous, black and thick paste.

The texture of this paste is correct for a mascara.

Regarding other application characteristics, the study via the user panel showed that the reference mascara is easily applicable, fixes well on the eyelashes and separates well from the latter (absence of stuck eyelashes), has a short drying time, and lengthens the eyelashes with a volumizing effect.

### Example 3: Composition and Properties of a Mascara Comprising a Volatile Mixture of Alkanes According to the Prior Art

In the composition of example 3 of a mascara according to the prior art, patent application EP3773432A1, the isododecane (CAS 13475-82-6) present in phase 1 of the reference formula was substituted by a volatile mixture of alkanes consisting of 15% n-decane (CAS 124-18-5) and 85% n-dodecane (CAS 112-40-3).

### Drying Time of Mascara According to the Prior Art

The mascara of example 3 obtained according to the protocol of example 1 has a drying time on the eyelashes equal to 5 min 30 sec.

This drying time is not satisfactory for a mascara formulation.

### Characteristics of Application of Mascara According to the Prior Art

To establish this study, the formulation of the mascara according to the prior art was tested by a panel of users.

Thus, according to this study, the mascara according to the prior art has a satisfactory appearance in the form of a smooth, homogeneous, black and thick paste.

The texture of this paste is correct for a mascara.

Regarding other application characteristics, the study via the user panel showed that the mascara is easily applicable, fixes well on the eyelashes and separates well from the latter (absence of stuck eyelashes).

However, this same study showed that the volumizing and eyelash lengthening properties were less satisfactory than those of the reference mascara formulation.

Thus, the mascara composition is not satisfactory compared with the reference mascara composition.

### Example 4: Composition and Properties of a Mascara According to the Present Invention

In the composition of Example 4 of a mascara according to the present invention, the isododecane (CAS 13475-82-6) present in phase 1 of the reference formula has been substituted by a volatile mixture of alkanes.

For this example, the volatile alkane mixture consists of 20% 2,3,6-trimethylheptane (CAS 4032-93-3) and 80% n-dodecane (CAS 112-40-3).

### Drying Time of Mascara from Example 4

The mascara of example 4 obtained according to the protocol of example 1 has a drying time on the eyelashes equal to 3 min 50 sec.

This drying time is correct for a mascara formulation.

### Application Features of Mascara from Example 4

To establish this study, the formulation of the mascara according to the invention was tested by a panel of users.

Thus, according to this study, the mascara in example 4 has a satisfactory appearance in the form of a very shiny, smooth, homogeneous, intense black and thick paste.

The texture of this paste is correct for a mascara.

As regards the other application characteristics, the study via the user panel showed that the mascara according to the present invention is easily applicable, fixes well on the eyelashes and separates well from the latter (absence of stuck eyelashes).

Thus, the mascara composition according to the invention is satisfactory.

### Example 5: Composition and Properties of a Mascara According to the Present Invention

In the composition of Example 5 of a mascara according to the present invention, the isododecane (CAS 13475-82-6) present in phase 1 of the reference formula has been substituted by a volatile mixture of alkanes.

For this example, the volatile alkane mixture consists of 20% 2,3,6-trimethylheptane (CAS 4032-93-3) and 80% C11-13 marketed as CETIOL Ultimate by BASF.

### Drying Time of Mascara from Example 5

The mascara, according to the invention of example 5 obtained according to the protocol of example 1, has a drying time on the eyelashes equal to 4 min 10 sec.

This drying time is correct for a mascara formulation.

### Application Characteristics of Mascara from Example 5

To establish this study, the formulation of the mascara according to the invention was tested by a panel of users.

Thus, according to this study, the mascara in example 5 has a satisfactory appearance in the form of a shiny, smooth, homogeneous, black and thick paste.

The texture of this paste is correct for a mascara.

As regards the other application characteristics, the study via the user panel showed that the mascara according to the present invention is easily applicable, fixes well on the eyelashes and separates well from the latter (absence of stuck eyelashes).

In addition, this study also showed that the mascara according to the invention has a satisfactory drying time, volumizing effect and eyelash lengthening.

It is noted that the mascara composition according to the invention is slightly granular compared with the reference formulation.

The mascara composition according to the invention is still considered satisfactory.

### Example 6: Composition and Properties of a Mascara According to the Present Invention

In the composition of Example 6 of a mascara according to the present invention, the isododecane (CAS 13475-82-6) present in phase 1 of the reference formula has been substituted by a volatile mixture of alkanes.

For this example, the volatile alkane mixture consists of 35% 2,3,6-trimethylheptane (CAS 4032-93-3) and 65% n-dodecane (CAS 112-40-3).

### Drying Time of Mascara from Example 6

The mascara according to the invention obtained according to the protocol of example 1 has a drying time equal to 3 min 00 sec.

This drying time is very satisfactory for a mascara formulation.

### Application Characteristics of Mascara from Example 6

To establish this study, the formulation of the mascara according to the invention was tested by a panel of users.

Thus, according to this study, the mascara of example 6 has a satisfactory appearance in the form of a shiny, smooth, homogeneous, intense black and thick paste.

The texture of this paste is correct for a mascara.

As regards the other application characteristics, the study via the user panel showed that the mascara according to the present invention is easily applicable, fixes well on the eyelashes and separates well from the latter (absence of stuck eyelashes).

Additionally, this study also showed that the mascara has satisfactory drying time, volumizing effect and eyelash lengthening.

The mascara composition according to the invention is very satisfactory.

### Example 7: Summary Table of the Different Tests of Examples 2 to 6 and Comparison with a Commercial Mascara

The table below is a comparison table of the different tests in examples 2 to 6.

A commercial mascara is also included in this table under the name Mascara Velvet Cushion 2 marketed by the company MAKE UP FOREVER.

| | | | Mascara | Mascara | Mascara | Mascara | Velvet Cushion |
|---|---|---|---|---|---|---|---|
| | | | of | according | according | according | 2 commercial |
| | | | the | to | to | to | mascara |
| | | Reference | prior | the | the | the | (MAKE |
| Volatile solvent | Example | mascara | art | invention | invention | invention | UP FOREVER) |
| Isododecane | 2 | 25.3% | — | — | — | — | |
| 15% n-decane | 3 | — | 25.3% | — | — | — | |
| 85% n-dodecane | | | | | | | |
| 20% 2,3,6-trimethylheptane | 4 | — | — | 25.3% | — | — | |
| 80% n-dodecane | | | | | | | |
| 20% 2,3,6-trimethylheptane | 5 | — | — | — | 25.3% | — | |
| 80% C11-13 alkane | | | | | | | |
| 35% 2,3,6-trimethylheptane | 6 | — | — | — | | 25.3% | |
| 65% dodecane | | | | | | | |
| Drying time on lashes | | 2 min | 5 min | 3 min | 4 min | 3 min | 4 min |
| | | 50 | 30 | 50 | 10 | 00 | 10 |

### Example 8: Composition of an Eyeliner According to the Present Invention

In order to prepare the eyeliner compositions according to the present invention, Examples 8 and 9, phase 1 is weighed and heated to a maximum temperature of 40° C.

Separately, phase 2 is weighed into another container and then phase 2 is heated to a temperature of 60° C. Then, after cooling, phase 2 is added to phase 1, as well as phase 3 in a second step.

Phase 4 is then added gradually with vigorous magnetic stirring to obtain an emulsion.

Finally, the powders from phase 5 are added to the mixture.

| | | | % by |
|---|---|---|---|
| PHASE | TRADE NAME | INCI | mass |
| 1 | Volatile mixture of alkanes according to the present | | 37.8 |
| | invention | | |
| | Vegelight Bentone | COCONUT ALKANES | 10 |
| | Silk SB | (AND) STEARALKONIUM | |
| | | BENTONITE | |
| | | (AND) TRIETHYL CITRATE | |
| | MI BLACK | ALUMINUM DIMYRISTATE | 14 |
| | | (AND) CI 77499 | |
| | OLIVE GEL SQE | DIMER DILINOLEYL | 5.5 |
| | | DIMER DILINOLEATE & | |
| | | OLIVE (OLEA EUROPAEA) | |
| | | OIL UNSAPONIFIABLES & | |
| | | DILINOLEIC ACID/ | |
| | | BUTANEDIOL COPOLYMER | |
| | BELSIL TMS 803 | TRIMETHYLSILOXYSILICATE | 3 |
| 2 | MYRITOL 318 | CAPRYLIC CAPRIC | 1.8 |
| | | TRIGLYCERIDE | |
| | VEGEWAX 55 | HYDROGENATED | 5 |
| | SUN | SUNFLOWER SEED OIL | |
| | BEESWAX | BEESWAX | 3 |
| | WHITE SP 422P | | |
| | CRODABOND | HYDROGENATED CASTOR | 1.5 |
| | CSA LQ | OIL/SEBACIC ACID | |
| | | COPOLYMER | |
| | Volatile mixture of alkanes according to the present | | 2 |
| | invention | | |
| 3 | MICROCARE PE | PHENOXYETHANOL | 0.2 |
| | DERMOSOFT | CAPRYLYL GLYCOL | 0.5 |
| | OCTIOL | | |
| 4 | DEIONIZED | WATER | 10 |
| | WATER | | |
| | POTASSIUM | POTASSIUM SORBATE | 0.2 |
| | SORBATE | | |
| 5 | SILICA BEAD SB | SB 700 SILICA | 2 |
| | 700 | SYNTHETIC | 2 |
| | MIYOSYN | FLUORPHLOGOPITE | |
| | MATTE-ALT | (AND) | |
| | | TRIETHOXYCAPRYLYLSILANE | |
| | SENSOSEL 10 | CELLULOSE | 1.5 |

The composition of the eyeliner of Example 8 prepared above comprises a volatile mixture of alkanes consisting of 40% 2,3,6-trimethylheptane (CAS 4032-93-3) and 60% n-dodecane (CAS 112-40-3).

This cosmetic formulation has an index of naturalness equal to 90% according to the ASTM D6866 standard.

In other words, this cosmetic formulation is made up of 90% ingredients of natural origin.

### Example 9: Composition of an Eyeliner According to the Present Invention

| PHASE | TRADE NAME | INCI | % |
|---|---|---|---|
| 1 | Volatile mixture of alkanes according to the present | | 37.8 |
| | invention | | |
| | Vegelight Bentone | COCONUT ALKANES (AND) | 10 |
| | Silk SB | STEARALKONIUM | |
| | | BENTONITE (AND) | |
| | | TRIETHYL CITRATE | |
| | MI BLACK | ALUMINUM DIMYRISTATE | 14 |
| | | (AND) CI 77499 | |
| | OLIVE GEL SQE | DIMER DILINOLEYL | 7 |
| | | DIMER DILINOLEATE & | |
| | | OLIVE (OLEA EUROPAEA) | |
| | | OIL UNSAPONIFIABLES & | |
| | | DILINOLEIC ACID/ | |
| | | BUTANEDIOL COPOLYMER | |
| | KAHLRESIN | SJOREA ROBUSTAE | 1.5 |
| | 6721 | RESIN & CERA ALBA | |
| 2 | MYRITOL 318 | CAPRYLIC CAPRIC | 1.8 |
| | | TRIGLYCERIDE | |
| | VEGEWAX 55 | HYDROGENATED | 5 |
| | SUN | SUNFLOWER SEED OIL | |
| | BEESWAX | BEESWAX | 3 |
| | WHITE SP 422P | | |
| | CRODABOND | HYDROGENATED CASTOR | 1.5 |
| | CSA LQ | OIL/SEBACIC ACID | |
| | | COPOLYMER | |
| | Volatile mixture of alkanes according to the present | | 2 |
| | invention | | |
| 3 | MICROCARE PE | PHENOXYETHANOL | 0.2 |
| | DERMOSOFT | CAPRYLYL GLYCOL | 0.5 |
| | OCTIOL | | |
| 4 | DEIONIZED | WATER | 10 |
| | WATER | | |
| | POTASSIUM | POTASSIUM SORBATE | 0.2 |
| | SORBATE | | |
| 5 | SILICA BEAD | SB 700 SILICA | 2 |
| | SB 700 | | 2 |
| | MIYOSYN | SYNTHETIC | |
| | MATTE-ALT | FLUORPHLOGOPITE (AND) | |
| | | TRIETHOXYCAPRYLYLSILANE | |
| | SENSOSEL 10 | CELLULOSE | 1.5 |

The composition of the eyeliner of Example 9 prepared above comprises a volatile mixture of alkanes consisting of 40% 2,3,6-trimethylheptane (CAS 4032-93-3) and 60% n-dodecane (CAS 112-40-3).

This cosmetic formulation has an index of naturalness equal to 93% according to the ASTM D6866 standard.

In other words, this cosmetic formulation is made up of 93% ingredients of natural origin.

### Example 10: Biodegradability of Volatile Mixtures of Alkanes Used in the Eye Makeup Compositions According to the Invention

The biodegradability of a volatile mixture of alkanes used in the eye makeup compositions according to the invention was measured according to the OECD 301F method. For comparison, the degradation of isododecane (CAS 13475-82-6) was also measured with this method.

| | Percentage of degradation |
|---|---|
| | according to the OECD |
| Substance | 301F method |
| Isododecane (CAS 13475-82-6) | 0% |
| Volatile mixture of alkanes: 35% 2,3,6- | 58% |
| trimethylheptane (CAS 4032-93-3) and | |
| 65% n-dodecane (CAS 112-40-3) | |

The OECD 301F method is used to characterize the ease of biodegradation of a substance by a manometric respirometry test.

The volatile mixture of alkanes tested shows a biodegradability of 58% according to the OECD 301F method while isododecane (CAS 13475-82-6) is not biodegradable according to this method.

1. An eye makeup composition wherein it comprises a volatile mixture of alkanes, the volatile mixture of alkanes comprising:
a) from 20 to 100% of a fraction A which comprises at least one branched C≤10 alkane;
b) from 0 to 80% of a fraction B which comprises at least one linear or branched C>10 alkane;
and wherein the volatile mixture of alkanes has a flash point below 55° C. measured according to ASTM D93 and represents a mass percentage greater than or equal to 10% relative to the total mass of the composition.

2. The eye makeup composition according to claim 1, wherein the volatile mixture of alkanes having a flash point below 55° C. measured according to standard ASTM D93 represents a mass percentage greater than or equal to 25% relative to the total mass of the composition.

3. The eye makeup composition according to claim 1, wherein the volatile mixture of alkanes has a percentage greater than or equal to 85% of carbon 14 (¹⁴C) determined according to one of the following standards ASTM D6866, EN 16640 or EN 16785-1.

4. the eye makeup composition according to claim 1, wherein the volatile mixture of alkanes is obtained from raw materials of plant, bacterial or animal origin.

5. The eye makeup composition according to claim 1, wherein the volatile mixture of alkanes has a biodegradability of at least 50% according to the OECD 301F method.

6. The eye makeup composition according to claim 1, wherein at least one branched C≤10 alkane of the volatile mixture of alkanes is chosen from the group comprising branched C₉ alkanes, branched C₁₀ alkanes, and mixtures thereof.

7. The eye makeup composition according to claim 1, wherein at least one branched C≤10 alkane of the volatile mixture of alkanes is a branched C₁₀ alkane.

8. The eye makeup composition according to claim 1, wherein at least one branched C≤10 alkane of the volatile mixture of alkanes is 2,3,6-trimethylheptane (CAS 4032-93-3).

9. The eye makeup composition according to claim 1, wherein the mass percentage of fraction A of the volatile mixture of alkanes is between 51% and 100% relative to the total mass of the volatile mixture of alkanes.

10. The eye makeup composition according to claim 1, wherein at least one linear or branched C>10 alkane of the volatile mixture of alkanes is selected from the group comprising C₁₁, C₁₂, C₁₃, C₁₄, C₁₅, C16 alkanes and mixtures thereof.

11. The eye makeup composition according to claim 1, wherein at least one linear or branched C>10 alkane of the volatile mixture of alkanes is n-dodecane (CAS 112-40-3).

12. The eye makeup composition according to claim 1, wherein the mass percentage of at least one linear or branched C>10 alkane of the volatile mixture of alkanes is between 0 and 50% relative to the total mass of the volatile mixture of alkanes.

13. The eye makeup composition according to claim 1, wherein the volatile mixture of alkanes comprises 2,3,6-trimethylheptane (CAS 4032-93-3) and n-dodecane (CAS 112-40-3).

14. The eye makeup composition according to claim 1, wherein it comprises at least one non-volatile oil having a flash point greater than 110° C. measured according to the ATSM D93 standard.

15. The eye makeup composition according to claim 1, wherein at least 85% by mass of its ingredients is of natural origin relative to the total mass of the composition.

16. The eye makeup composition according to claim 1, wherein the drying time at 20° C. after application of the composition to the eyelashes is less than or equal to 4 min.

17. A process for preparing an eye makeup composition according to claim 1, comprising the following steps:
a) production of a C10 Guerbet alcohol, 1-Hexanol, 2-isopropyl-5-methyl (CAS 2051-33-4) by Guerbet synthesis from isoamyl alcohol (CAS 123-51-3);
b) the mixture of the product formed in step 1) with at least one bio-sourced alcohol in C>10 to form a bio-based alcohol mixture;
c) obtaining a volatile mixture of alkanes consisting of 2,3,6-trimethylheptane (CAS 4032-93-3) and a C alkane>10, by a dehydration reaction followed by a hydrogenation reaction of the bio-sourced alcohol mixture formed in step 2);
d) the addition of none, one or more volatile alkanes to this volatile mixture of alkanes;
e) the combination of this volatile mixture of alkanes with at least one compound chosen from non-volatile oils, glossy oils, pasty fatty substances, gelling agents, film-forming polymers, waxes, pigments, dyes, and mixtures thereof.
